(19) United States
(12) Patent Application Publication
(10) Pub. No.: US 2014/0257075 A1
(43) Pub. Date: Sep. 11, 2014

(54) MEASURING AQUEOUS HUMOR OUTFLOW

(71) Applicants: Lawrence E. Kagemann, JR., Pittsburgh, PA (US); Joel S. Schuman, Pittsburgh, PA (US)

(72) Inventors: Lawrence E. Kagemann, JR., Pittsburgh, PA (US); Joel S. Schuman, Pittsburgh, PA (US)

(73) Assignee: University of Pittsburgh - Of The Commonwealth System of Higher Education, Pittsburgh, PA (US)

(21) Appl. No.: 14/236,600

(22) PCT Filed: Aug. 1, 2012
(86) PCT No.: PCT/US2012/049136

§ 371 Date: May 7, 2014

Related U.S. Application Data
(60) Provisional application No. 61/514,689, filed on Aug. 3, 2011.

(51) Int. Cl. A61B 3/16; A61B 3/00; A61B 3/10

(52) U.S. Cl. CPC A61B 3/165; A61B 3/102; A61B 3/0025
USPC 600/399

(57) ABSTRACT

Technologies are provided to estimate in vivo aqueous humor outflow for a subject. The method can include: preparing a virtual casting of the outflow network of the subject by use of background subtraction and contrast enhancement; tracing network terminal branches in said reduced virtual casting; obtaining Doppler data for at least some of the terminal branches, to calculate a fluid velocity within each of such terminal branch; then pairing each fluid velocity for each such terminal branch with a measurement of cross-sectional area for that terminal branch, thereby to provide a plurality of volumetric flow values; and integrating over the plurality to obtain a volumetric estimate of aqueous humor outflow for the subject.

### CROSS-REFERENCE TO RELATED APPLICATIONS

This application claims priority to U.S. Provisional Patent Application No. 61/514,689, filed on Aug. 3, 2011, the entirety of which is incorporated by reference.

### STATEMENT OF GOVERNMENT INTEREST

The present invention was made with government support under grants No. EY013178, No. EY009098 and No. EY013516, awarded by the National Institutes of Health. The government has certain rights in this invention.

### BACKGROUND OF THE INVENTION

Glaucoma is the second leading cause of irreversible blindness, reducing quality of life and increasing healthcare costs for glaucoma patients (Kymes et al.; Quigley and Broman, 2006). The greatest risk factor for the presence and progression of glaucoma is elevated intraocular pressure or IOP (Dielemans et al., 1994; Kahn et al., 1977; Kass et al., 1980; Reynolds, 1977; Sommer, 1989; Vacharat, 1979). IOP is regulated by a balance between the production and drainage of aqueous humor (AH) (Duke-Elder, 1949; Millar and Kaufman, 1995).

Most AH leaves the eye through the trabecular meshwork, in the angle of the anterior chamber, and through Schlemm's canal (SC). AH leaves SC either via collector channels to a complex network of aqueous venous plexuses, including the deep, midlimbal and perilimbal scleral plexuses, ultimately draining into scleral veins or Ascher's aqueous veins, which connect directly from SC to the episcleral veins (Ascher, 1942; Ashton, 1951, 1952; Ashton and Smith, 1953; van der Merwe and Kidson, 2010).

### SUMMARY OF THE INVENTION

The inventors have developed a technique for the noninvasive visualization and quantification of the primary pathway of aqueous humor outflow in the human eye. Pursuant to this technique, volumetric circumferential scans of the limbus are obtained. To this end will suffice any scan that contains structure and Doppler data, including but not limited to scans performed via spectral domain optical coherence tomography (SD-OCT), as exemplified below. Scan data can be adjusted such that the associated gray-scale presentation features outflow vessels as white structure on dark background. A rolling ball background subtraction algorithm then is applied, and contrast is adjusted to isolate the outflow vessels. Individual processed volumes are stitched together to provide a perilimbal view of outflow structures. Terminal branches in the outflow vascular network are identified, and Doppler is measured within those structures. Doppler and cross-sectional assessments are combined to calculate flow in each terminal branch of the outflow network. Total aqueous humor outflow then can be determined by integrating flow across all identified terminal outflow structures. Thus, the invention provides a direct, noninvasive measurement of aqueous outflow in the primary outflow pathway.

Pursuant to certain embodiments of the invention, a series of filters initially is applied, in order to isolate and to visualize aqueous humor outflow structure within the eye as a virtual casting, i.e., in three dimensions. Such virtual castings can be utilized to discriminate between terminal and redundant sources of Doppler measurements in the functional anterior segment imagery. In accordance with another aspect of the invention, moreover, a subset of locations necessary to measure total aqueous outflow can be identified without redundant measurements of overlapping vessels.

Accordingly, methodology, apparatus, systems, and computer software are provided to estimate in vivo aqueous humor outflow for a subject. A method of the invention can include preparing a virtual casting of the outflow network of said subject by use of background subtraction and contrast enhancement; tracing network terminal branches in said reduced virtual casting; obtaining Doppler data for at least some of said terminal branches, to calculate a fluid velocity within each of such terminal branch; then pairing each fluid velocity for each such terminal branch with a measurement of cross-sectional area for that terminal branch, thereby to provide a plurality of volumetric flow values; and integrating over said plurality to obtain a volumetric estimate of aqueous humor outflow for said subject.

All combinations of the foregoing concepts and additional concepts discussed in greater detail below, provided such concepts are not mutually inconsistent, are contemplated as being a part of the inventive subject matter described here. In particular, all combinations of claimed subject matter appearing at the end of this disclosure are deemed part of the described inventive subject matter. Terminology explicitly employed here that also may appear in any disclosure incorporated by reference should be accorded a meaning most consistent with the concepts particularly described here.

### BRIEF DESCRIPTION OF THE DRAWINGS

The foregoing and other aspects, embodiments, and features of the present invention can be understood more fully from the following description, in conjunction with the accompanying drawings, which are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, with emphasis instead on illustrating principles of the invention. The drawings are not intended to limit the scope of the invention.

As FIG. 1 shows, SD-OCT and ultrasound imaging of the anterior segment have produced cross-sectional images of the drainage system, but these do not yield sufficient visualization to ascertain the condition or density of the complex three-dimensional (3D) structures of the AH outflow system. See Irshad et al., 2010, Kagemann et al., 2010, and Sarunic et al., 2008.

Aqueous humor (AH) exiting the eye via the trabecular meshwork and Schlemm's canal (SC) passes through the deep and intrascleral venous plexus (ISVP) or directly through aqueous veins. Pursuant to the present invention, the human AH outflow system can be visualized, in a virtual casting, in 360 degrees in three dimensions (3D) during active AH outflow. Also, the invention will permit the AH outflow pathways to be imaged in vivo among patients.

In an illustrative experiment, the conventional AH outflow pathways of seven donor eyes were imaged with an SD-OCT system, i.e., an SD-OCT device manufactured by Bioptigen Inc. (Research Triangle Park, N.C., USA) and, as a light source, a quad diode array manufactured by SuperLum Ltd. (Dublin, Ireland), at a perfusion pressure of 20 mm Hg (N=3) and 10 mmHg (N=4). In these eyes, thirty-six scans (three equally distributed in each clock hour), each covering a 2×3×2 mm volume (512 frames, each 512×1024 pixels), were obtained. All image data were black/white inverted and the background was subtracted, using the Image Processing and Analysis in Java tools—ImageJ, provided on the NIH website. Contrast was adjusted to isolate the ISVP.

Observed as a result throughout the limbus were SC, collector channels, the deep and ISVP, and episcleral veins. Aqueous veins could be observed extending into the episcleral veins. Individual scan ISVP castings were rendered and assembled in 3D space in Amira 4.1 (Visage Imaging Inc., USA). A 360-degree casting of the ISVP was obtained in all perfused eyes (see, e.g., FIG. 6). The ISVP tended to be dense and overlapping in the superior and inferior quadrants, and thinner in the lateral quadrants.

In accordance with some embodiments of the invention, imaging of the human AH outflow pathway can be accomplished using, for example, SD-OCT as discussed above. The more superficial structures of the AH outflow pathway present with sufficient contrast as to be optically isolated and cast in-situ 360 degrees in cadaver eye perfusion models. This approach will be useful for studying human AH outflow, e.g., in diagnostic and prognostic contexts.

FIG. 1 illustrates that the anterior chamber angle is scanned by SD-OCT. (A) Scans contain portions of the iris, cornea, and limbus. Within the scan (B) Schlemm's canal and aqueous veins, and the intrascleral venous plexus (ISVP) are seen.

FIG. 2 illustrates that spectral domain optical coherence tomography images would normally be displayed with a low level of brightness. (A) In the case of highly averaged image data (700 axial scans—A-scans—in this frame; each A-scan the average of 18 sequentially acquired A-scans), brightness can be increased (B) allowing visualization of deep low signal structures such as the iris, angle, trabecular meshwork, and endothelial/Descemet's complex without compromising due to visible noise. SDOCT images obtained without averaging (512 A-scans, each presented without averaging) and displayed with a low level of brightness (C) have an appearance similar to those obtained with averaging at the same noise suppression level. Yet, increasing brightness reveals the noise masking the deep layers where the signal is low (D).

FIG. 3 illustrates that the averaged image stack has a spatial resolution of 512×512×1024 voxels encompassing a physical volume of 2×3×2 mm. (A) A single averaged frame. (B) The data were resampled to yield a 1:1:1 aspect ratio. The resampled data were inverted (C) and background was subtracted. (D) Contrast was then adjusted to isolate the structures of interest (E).

FIG. 4 illustrates that the isolated intrascleral venous plexus image data can be rendered in 3D to visualize the ISVP during active outflow. This is one of 36 circumferential scans of the limbus, showing a portion of the venous network. The red marker on the right indicates the X, Y, and Z axes of the 3D space, with the interrogating SDOCT beam projecting down the Z axis, and raster scanning the X-Y plane.

FIG. 5 shows that, to reveal the posterior surface of the vascular network, a single slice is rotated and seen from three perspectives. Aqueous veins thus could be observed penetrating the intrascleral venous plexus from proximal structures in the aqueous humor outflow system. (A) A virtual casting from the 6:00 position in an eye perfused at 20 mm Hg can be rotated (B) to expose the posterior surface (C) revealing two aqueous veins (white arrows).

FIG. 6 depicts the results when 36 circumferential limbal scans were processed and assembled manually in 3D space to yield a full casting of the episcleral and intrascleral venous plexus throughout the limbus in-situ, during active perfusion, according to the invention.

FIG. 7(A)-(J) illustrates an image-processing method according to the invention.

FIG. 8(A)-(L) illustrates an image-processing technique, in accordance with the invention, for automatic identification of movement in noisy Doppler images.

FIG. 9 illustrates a virtual casting of a living human eye that was obtained noninvasively, in accordance with an embodiment of the invention.

FIG. 10 shows imaging via an implementation of an embodiment of the invention on the Cirrus HD-OCT system (Carl Zeiss Meditec, Dublin, Calif.). The imaging of the limbus (left) clearly reveals Schlemm's canal (black arrows), as well as a distinctive “fallen Y” aqueous vein. The characteristics of this structure was used to locate the same cross-sectional image within image data (right) obtained, pursuant to the invention, with a Bioptigen SDOIC system (Bioptigen, Research Triangle Park, N.C.). Two distinct layers of aqueous venous plexuses are visible (white block arrows, right), at two depths within the limbus (white block arrows, left). Unlike blood vessels (stars), which cast vertical shadows into the scan (Xs), aqueous veins do not create shadowing artifacts in the image (white bar=1 mm).

FIG. 11 depicts two virtual castings of Schlemm's canal that were produced from volumetric anterior segment scans of the limbus obtained with the above-mentioned Cirrus system. Schlemm's canal is marked with asterisks, and locations of connecting collector channels at ostia are marked with arrows.

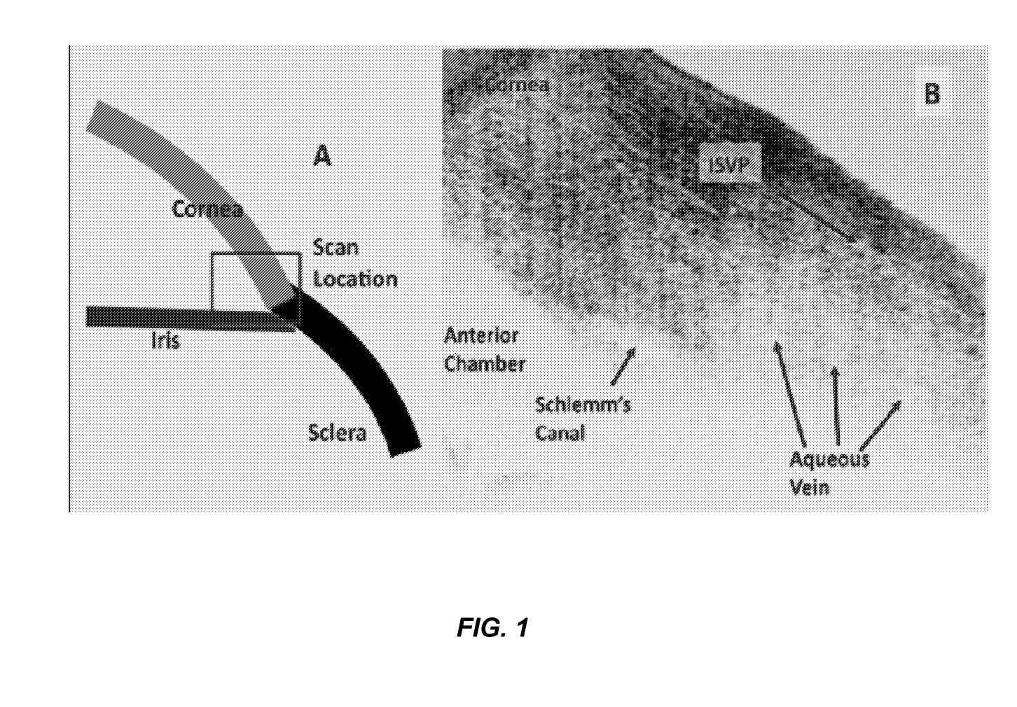

### DETAILED DESCRIPTION OF THE INVENTION

The following is a more detailed description of various concepts related to and of embodiments of inventive methodology and apparatus for visualizing and quantifying AH outflow. The various concepts introduced above and further discussed here may be implemented in any of numerous ways; hence, the concepts are not limited to any particular manner of implementation. Examples of particular implementation and application are provided primarily for illustrative purposes.

SD-OCT rapidly quantifies tissue reflectance in 3D cubes at speeds up to 512,000 A-scans per second. For instance, see Rollins et al., 1998, and Zhang and Kang, 2010. Coupling the high scanning speed with ultrahigh resolution, it is possible to visualize the individual components of the AH outflow system from the anterior chamber throughout the system of aqueous veins in the living human eye (Kagemann et al., 2010). However, shadows from superficial structures may obscure the deeper structures (id).

Superficial outflow structures, specifically the ISVP and episcleral veins, are readily visualized by SD-OCT. Embodiments disclosed herein provide a method for visualizing the 3D structures of the conventional AH outflow system in human cadaver eyes during perfusion with SD-OCT. After imaging, these same eyes were processed and examined by light microscopy for correlative histology.

In one experiment, human cadaver eyes with no history of eye disease, trauma or ocular surgery other than cataract were obtained from the Florida Eye Bank (Miami, Fla.), and the Center for Organ Recovery and Education (Pittsburgh, Pa.). The Committee for Oversight of Research Involving the Dead of the University of Pittsburgh approved the study. Consent for the use of all tissues for research was obtained by the individual agency responsible for harvesting and supplying the tissue.

To prepare for perfusion, seven eyes (Table 1) were wrapped in saline-soaked gauze, submerged in normal saline, and warmed to 40° C. Eyes were then placed in front of the SD-OCT scanner in a custom-made fixation mount. Throughout the experiment, the eye was irrigated with 40° C. saline to prevent dehydration and to minimize cooling. A 27-gauge needle was inserted into the peripheral cornea, with the needle tip passing through the pupil and positioned posterior to the iris and anterior to the lens. This positioning prevented artificial deepening of the anterior chamber during perfusion and artifactual increases in outflow facility (Ellingsen and Grant, 1971). Barany's mock AH (Barany, 1964) was used to perfuse the eyes. The initial 20 minutes of perfusion was used to establish baseline outflow. The rate of perfusion was determined by recording the weight of the AH in the reservoir in real time, 20 measurements per second. Measurements were recorded by a 4-channel, 10-bit digital acquisition system (DATAQ Instruments, Akron, Ohio). Immediately after completion of the perfusion experiments, the eyes were perfusion fixed with 10% formalin buffered solution before further processing for histological evaluation.

As detailed in the table below, seven eyes were imaged. The presence of the superficial tissues and anterior chamber pressure varied.

| Eye | Condition | Anterior Chamber Pressure |
|---|---|---|
| 1 and 2 | Intact | 20 mmHg |
| 3 | Conjunctiva and Tenon's | 20 mmHg |
| | Capsule Removed | |
| 4-7 | Conjunctiva and Tenon's | 10 mmHg |
| | Capsule Removed | |

Perfusion pressure is the hydrostatic force between the anterior chamber pressure and the pressure within the vessels receiving AH outflow. In this study, a normal episcleral venous pressure of 8 mmHg in living eyes was assumed (Erickson-Lamy et al., 1991). The initial pair of intact eyes was perfused with an anterior chamber pressure of 20 mmHg. Since the episcleral venous pressure in a cadaver eye is approximately 0 mmHg, an anterior chamber pressure of 20 mmHg produced a perfusion pressure equivalent to an IOP of 28 mmHg in a living eye. An anterior chamber pressure of 10 mm Hg yielded a perfusion pressure equivalent to an IOP of 18 mmHg in a living eye.

In the cadaver model, there is no active circulatory system present to remove AH expelled from the outflow system. See Kagemann et al., 2010. Fluid gradually accumulates in the conjunctiva and Tenon's capsule when that tissue was left intact on the globe, causing shadows obscuring visualization of outflow structures. Before perfusion, therefore, cadaver eyes require the removal of these layers in order to produce images of equal quality to those obtained in unperturbed living eyes. The conjunctiva and Tenon's capsule were dissected in all but the first pair of eyes.

Four eyes were perfused and imaged at 10 mmHg, and then perfusion fixed at 10 mm Hg for histology. One of these eyes was first imaged at a perfusion pressure of 0 mmHg. One eye was perfused and imaged at 20 mm Hg. After imaging, it was perfusion fixed at 20 mm Hg.

### SD-OCT Imaging

An SD-OCT optics engine (Bioptigen, Research Triangle Park, N.C.) was coupled with a high bandwidth superluminescent diode array (870 nm center wavelength, 200 nm bandwidth; model Q870, Superlum Ltd, Dublin, Ireland). This light source has a coherence length of 1.3 μm in tissue.

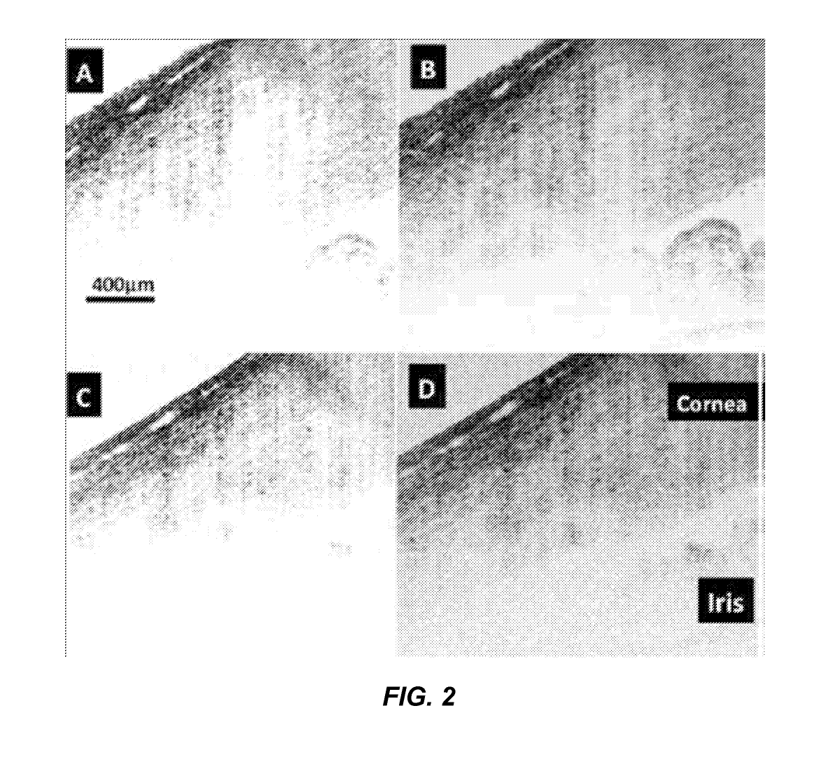

The optics engine allows the user to specify any number of A-scans per frame, and any number of frames, limited only by system memory. It also allows the user to specify any number of sequential A-scans to be acquired in a single location during a raster scan for the purpose of averaging and Doppler assessment, limited only by system memory. Two-scan protocols were created: one optimized for the acquisition of 3D data (the “volume” protocol) and one optimized for visualization of individual frames (the “2D slice” protocol). Each eye was scanned twice at the limbus, first with a protocol optimized for 3D volumes, and the second with a protocol optimized for visualization of 2D slices. Each set of images include 36 individual radial scan sets; each clock hour imaged at its center, and offset to the left and right. The angle of each set of clock hour scans was set so that the center clock hour scan was on a radial line from the center of the pupil (i.e. the 9 o'clock scan was at 0°, the 10 o'clock scan at 30°, the 11 o'clock scan at 60°, etc.). The 3D volume scan protocol was limited by system memory, and includes 512×512 A-scans probing a 2×3 mm (radial x transverse) area of tissue (FIGS. 2C and D).

This scan protocol moved the 20 μm-diameter SD-OCT beam 9 μm between A-scans, thus including a single tissue volume in multiple adjacent samples (oversampling). Acquiring oversampled SD-OCT data allowed post-process averaging. The 2D slice protocol includes 700×20 A-scans probing the same sized (2×3 mm) area of tissue. Each A-scan of the 2D slice imaging protocol was repeated 18 times, and the average of those 18 scans recorded (FIGS. 2A and B). This method was previously described in detail (Cense et al., 2006).

### Correlative Light Microscopy Imaging

After imaging using SD-OCT, two eyes were perfusion-fixed at 10 mmHg and one eye was perfusion fixed at 20 mm Hg. Following perfusion fixation, the three eyes were placed in 10% formalin buffered solution overnight and then transferred to PBS. The anterior chamber of each eye was cut radially into 12 pieces (1 clock hour each) and processed for light microscopic examination. The sections were post-fixed with 2% osmium tetroxide (Electron Microscopy Sciences, Hatfield, Pa.) in 1.5% potassium ferrocyanide (Fisher Scientific, N.J.) for 2 hours, dehydrated in a graded series of ethanols, and embedded in Epon-Araldite (Electron Microscopy Sciences, Hatfield, Pa.). Sections of 3 μm were cut (four blocks each eye at 3, 6, 9 and 12 o'clock) and stained with 1% Toluidine Blur (Fisher Scientific, N.J.). Light micrographs were taken at an original magnification of 4× and 10×. The histological images were compared with SD-OCT images from the same locations.

### SD-OCT Image Processing

Raw SD-OCT scan data are analyzed by histogram. The 75% of SD-OCT data with the lowest reflectance values are set to 0 when displayed (Stein et al., 2006). This approach is effective for the subjective improvement of visualization of highly reflective structures, but if the structures are in a region of low signal strength, they will not be displayed. In the slice image set, averaging during image acquisition improved visualization of structures with low reflectance. Increasing image brightness in highly averaged image data further improves visualization of the deep outflow structures (FIG. 2). In the volume image data set, however, raw data acquired from deep outflow structures were difficult to visualize. Increasing image brightness increases visualization of deep low-reflectance structures but also increases the appearance of speckle noise, obscuring structure details (FIG. 2). This can be overcome by signal averaging. The volume image protocol spatially oversamples the tissue, allowing averaging with minimal loss of structural information. A floating 5×5 pixel transverse kernel, including five pixels in each of five adjacent frames, was employed to produce an average dataset. Visualization of deep layers within the post-processing averaged volume dataset was comparable to that of the slice data (FIG. 2B).

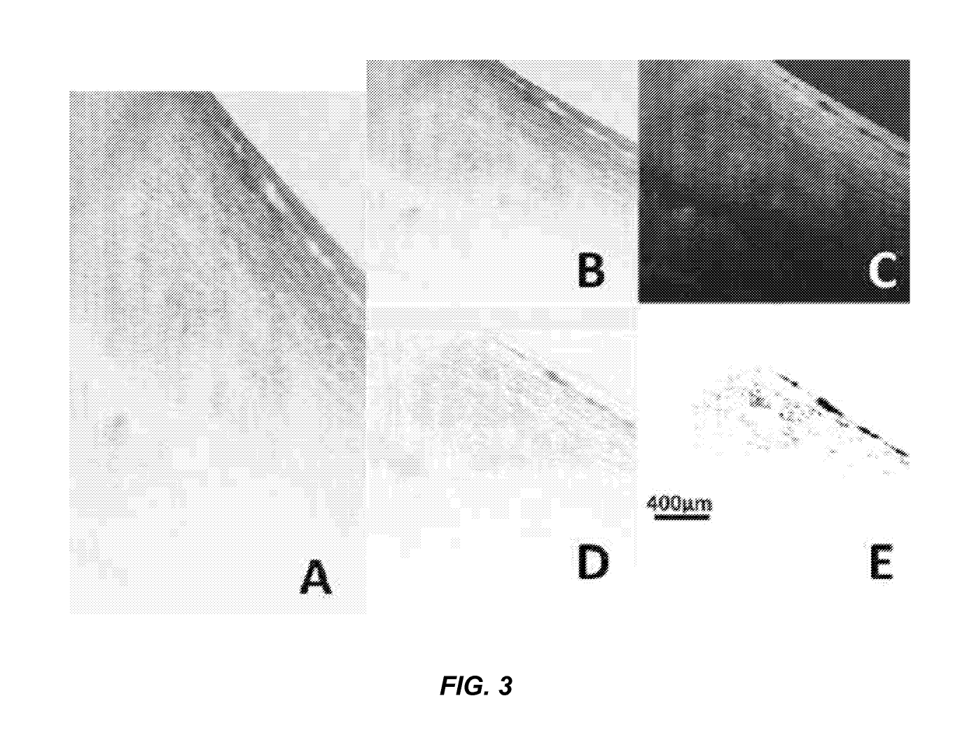

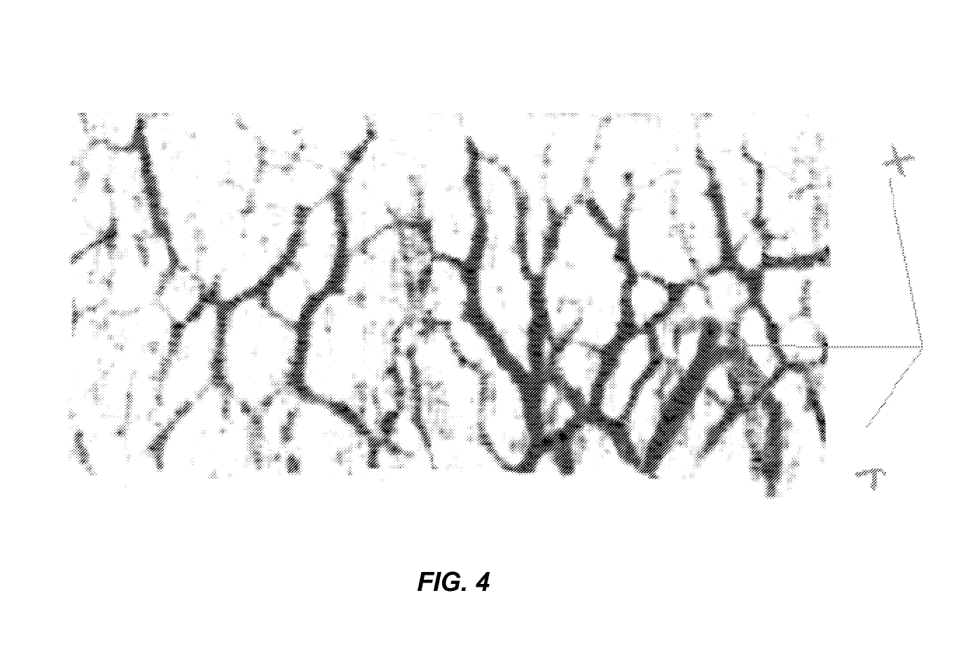

Further image processing was performed using ImageJ 64. Images were resampled to provide a 1:1:1 voxel aspect ratio in three dimensions; from 512×512×1024 (FIG. 3A) to 342×512×342 (FIG. 3B) for each 2×3×2 mm volume. Images then were inverted so that the black collector channels appeared as white structures (FIG. 3C). The ImageJ “subtract background” filter was applied with a 30 pixel kernel and without further averaging (FIG. 3D). Contrast then was adjusted to isolate the collector channels (FIG. 3E), and the volumes were rendered using the ImageJ 64 3D viewer plug-in (FIG. 4).

Individual stacks were opened in Amira 4.1 (Visage Imaging Inc., San Diego, Calif.) and rendered in 3D space with the Voltex module (2D texture, rendering downsample 3,3,3). Stacks were manually assembled in 3D space by overlaying aqueous veins and structures visible in adjacent scans. The volume scan protocol provided a large degree of overlap; most individual aqueous veins were contained in two images, and occasionally in three.

Outflow structures from the trabecular meshwork through the CC could be visualized throughout the limbus. The slice imaging protocol provided better visualization of outflow structures in cross-section, likely due to the combined effects of spatial oversampling (700 A-scans per frame) and aggressive averaging (18 sequentially acquired A-scans averaged to produce each displayed A-scan; FIG. 2B). Scanner memory restrictions may limit the number of frames that can be acquired using this protocol. The low number of frames makes the slice protocol unsuitable for enface imaging (FIG. 2D) or 2D reconstructions. The volume imaging protocol provides excellent enface images, and post-processing averaging allows the recovery of detail within deep structures. The sampling density of the volume protocol also allows high-resolution 3D reconstruction of the outflow system within individual scans (FIG. 4).

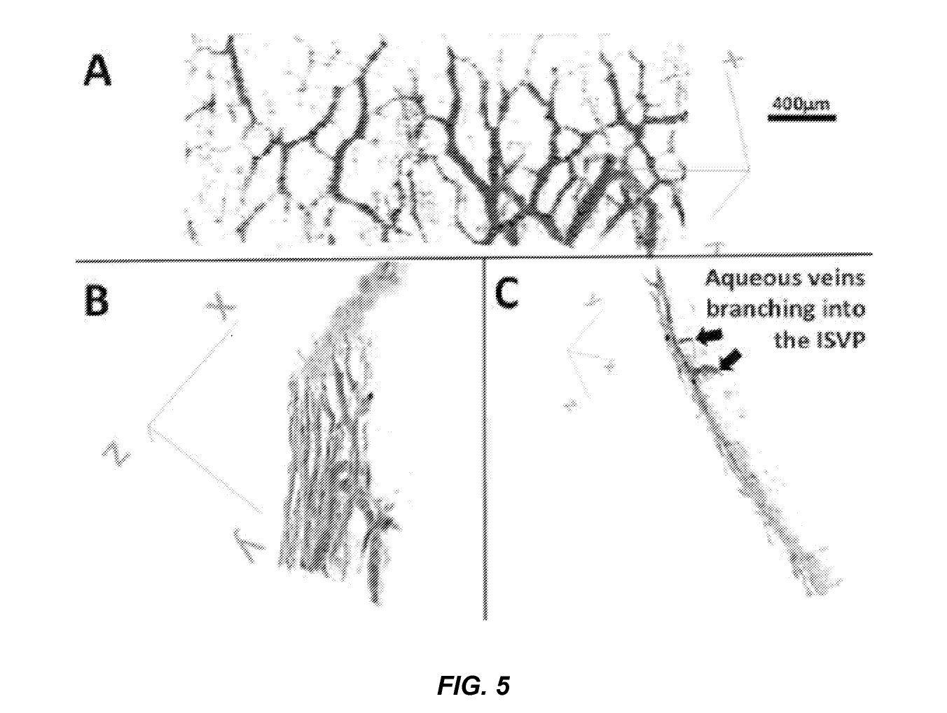

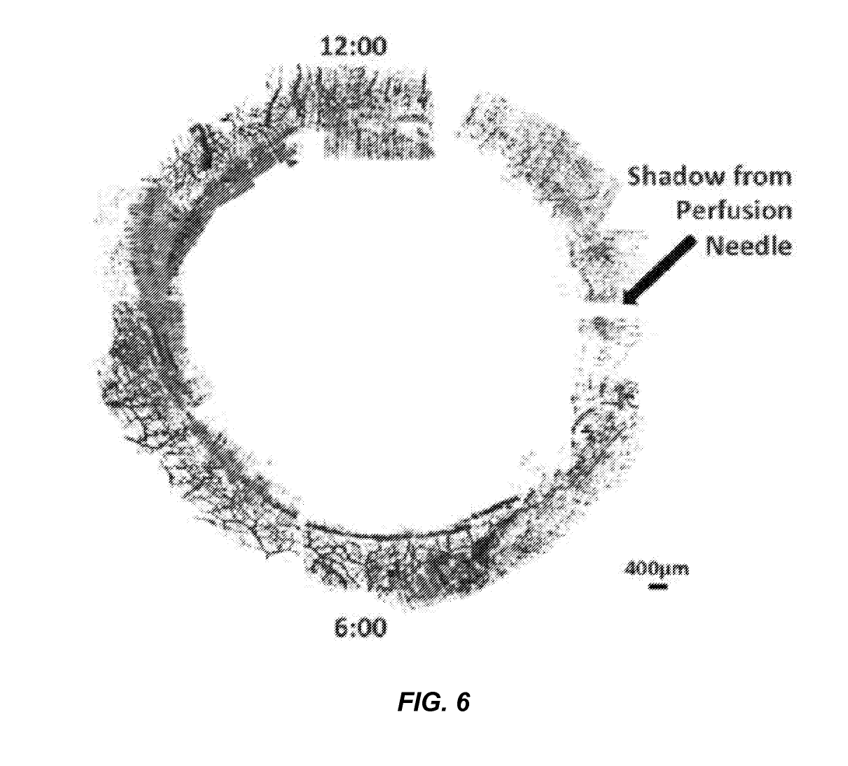

Exposure to elevated IOP may lead to closure of SC as the TM pushes and compresses the inner wall towards the outer wall (Battista et al., 2008). At 20 mm Hg perfusion pressure, SD-OCT revealed very few locations with a visible patent SC. This finding was confirmed by histology. Collector channel ostia and patent aqueous venous structures were observed by SD-OCT and confirmed by histology. At 10 mmHg, SC was not compressed. Smaller scleral veins running from the ISVP down toward the deep scleral venous plexus were frequently observed in individual frames of the 3D datasets, but seldom achieved sufficient contrast, relative to background tissue, to be observed in the 3D reconstructions. Occasionally, a large aqueous vein could be observed, but only when isolated from other branches of the ISVP. FIG. 5 shows the front and side views of a scan obtained from one eye, with the vessels evident that penetrate perpendicular to the ISVP. After individually processing the thirty-six scans of the limbus of the structural imaging protocol, a composite casting of the collector channels of the AH outflow system was rendered (FIG. 6). The densest arrays of aqueous veins within the ISVP were observed near the 6 and 12 o'clock positions.

Virtual casting of superficial venous plexus of AH outflow system is realized using methods and systems described here. Virtual casting takes advantage of the high degree of contrast between the superficial venous plexus of AH outflow system, including aqueous veins, and surrounding tissues achieved by aggressive post-processing averaging.

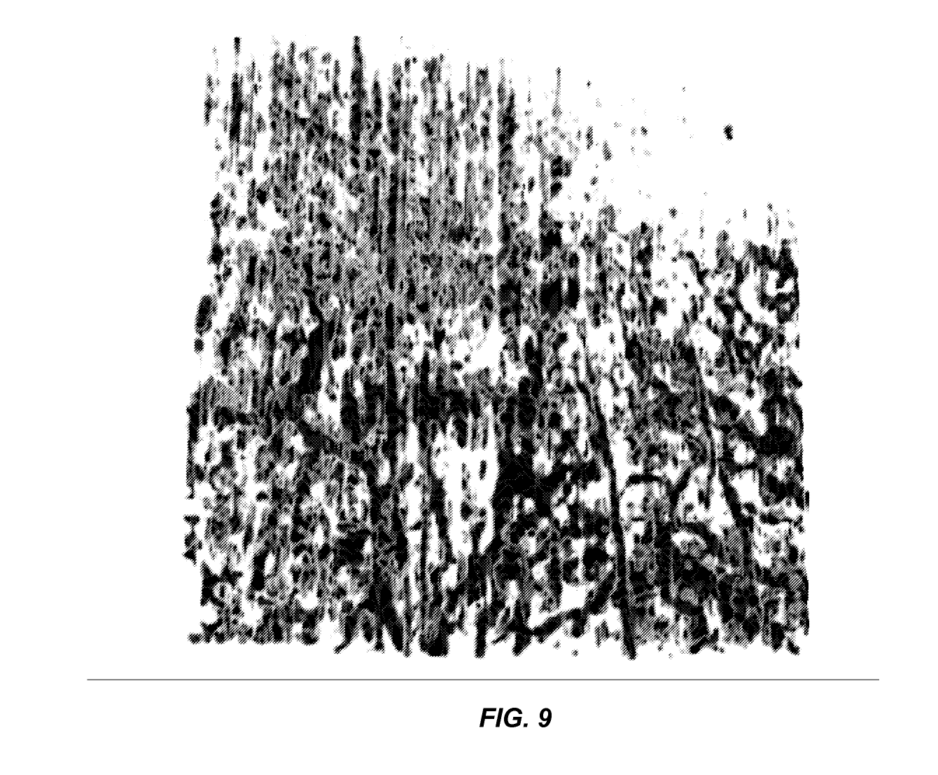

The imaging process can be non-contact, and dyes or contrast agents are not necessary. In the cadaver model, the conjunctiva and Tenon's capsule must be removed to produce images of similar quality as those produced in living human eyes, in relation to which the imaging process can be completely noninvasive. By way of illustration, FIG. 9 depicts a virtual casting from a living human eye, obtained via noninvasive methodology in accordance with an embodiment of the invention.

The inventors observed a good agreement between features in the SD-OCT 2D scans and the corresponding histological sections. This included the appearance of SC under different perfusion pressure conditions as well as the presence or absence of the open superficial vessels comprising the ISVP.

The cadaver eye used in the aforementioned experiment differs from a living eye. In particular, the cadaver eye lacks circulatory-related pulsations in IOP and blinking, each of which may contribute force to outflow (Johnstone, 2004). For instance, the change from a pulsatile to non-pulsatile environment might alter the conditions dictating the preferential location of outflow within the eye. There also is a lack of pulsatility in the vessels receiving AH. In the living eye, moreover, scleral veins receive AH in an environment of transient pressure waves. By contrast, in the cadaver eye AH arrives in vessels with no backpressure. These differences notwithstanding, the inventors determined that, in terms of subjective comparison of the quality of the cross-sectional OCT images, the quality of the castings those images produced, and the quality and magnitude of Doppler signals within, the cadaver outflow model provided an suitable foundation for elaborating the methodology of the present invention.

During perfusion the vessels were observed to fill with AH, resulting in some small constant backpressure. Combined with gravity, it is possible that the preferential outflow path in a cadaver model differs significantly from that of a living eye. Yet, were gravity the only influence then one would have expected full aqueous vessels in the inferior with a gradual diminution of the casting until it appeared empty in the superior. The inventors found the fullest ISVP castings in the superior and inferior quadrants. In living human eyes, SC near CC ostia had appeared to be larger in the nasal quadrant of the limbus (Kagemann et al., 2010).

As noted, the inventors' data indicated that the conjunctiva and Tenon's capsule needed to be removed before perfusion in order to image the deep layers of the limbus with quality equal to that obtained in living human eyes and to avoid distention of the outer tissue layers as they filled with AH. It is likely that the superficial-most vessels of the outflow system also were removed.

In the remaining tissues, it typically is infeasible to determine what percentage, if any, of the virtual casting consisted of ISVP or the midlimbal intrascleral plexus, or of some combination therein. Interconnectivity was observed within the vascular network. The lack of blood flow indicates that the casting is of active AH outflow. The identity of the actual observed vessels, whether aqueous vessels or venules, can be inferred by their location and connectivity. Some portions of the castings consisted of aqueous veins, as those portions were observed to penetrate into the limbus (FIG. 5), which could be seen in individual frames to extend directly to SC ostia. Nevertheless, since there was no active cardiac circulation in the imaged tissues, all passages captured in the casting were open and connected because they were carrying mock AH as part of the outflow process.

It would be desirable to have a complete casting of the outflow system, down to SC. That this is feasible is evidenced by a study that used micro CT to produce virtual castings of the outflow system at the level of SC in sections of a stained eye (Hann et al., 2011). See also Working Example 2, below.

In Working Example 1, only the superficial venous plexus or occasional large penetrating aqueous veins were isolated optically from surrounding tissues, although this was accomplished non-invasively and without introduction of any contrast agents. Pursuant to the invention, however, a more sophisticated image processing technique could include more structures in the casting. Thus, pre-processing with a contrast limited adaptive histogram equalization, followed by imposition of a connectivity requirement for inclusion in the casting, could be expected to reduce the use of contrast for isolation and allow inclusion of the smaller structures, weak signal levels notwithstanding.

In imaging AH outflow as it occurs, one goal is to be able to detect deficits associated with disease and to determine the effects of glaucoma medications and surgical interventions on outflow and the associated structures. Currently the two largest impediments to clinical implementation are penetration and eye movements. The structural and functional scan protocols exemplified here required approximately 10 seconds each, with a data acquisition rate of 28,000 A-scans per second. This may be unduly slow for obtaining useful data in human eyes, since eye movement artifacts are readily visible in 2 second scans obtained with commercially available SD-OCT's. Nevertheless, SD-OCT systems are available that have achieved a scan rate of 512,000 A-scans per second (Zhang and Kang, 2010), which would reduce the scan time to 0.5 seconds. Furthermore, despite the limits of the 870 nm-centered system as exemplified, the use of aggressive averaging allowed for the resolution of the structures of the angle and conventional outflow system (FIG. 2). Longer wavelength systems also may overcome limitations in penetration.

Clinical and research use will require the development of meaningful parameters that quantify outflow structures. The outflow structures are too numerous and dense, with marked regional variation, to allow for arbitrarily choosing individual CC's to represent outflow. Thus, assessment of the outflow venous network will involve an automated quantification of aqueous vein density and the distribution of vein sizes. Quantification of the number of branch points also may have some clinical meaning The determination of how each of these potential parameters is affected by the presence of glaucoma will aid the diagnosis of glaucoma. Accordingly, outflow casting analysis software in accordance with the invention will be used in conjunction with the scan instruments to form a diagnostic system.

### WORKING EXAMPLE 1

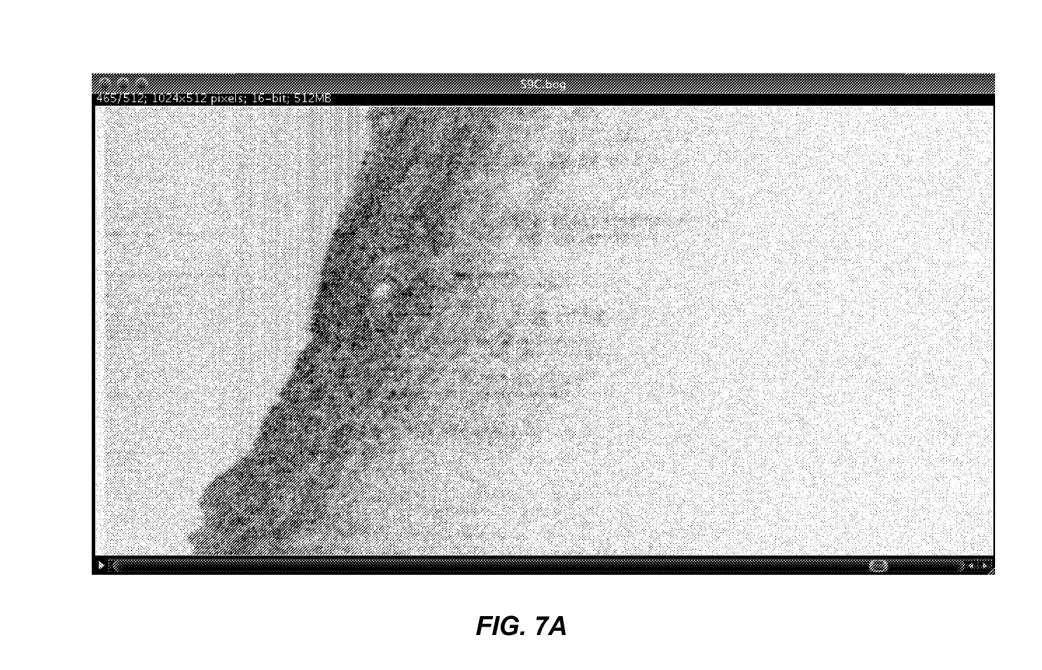

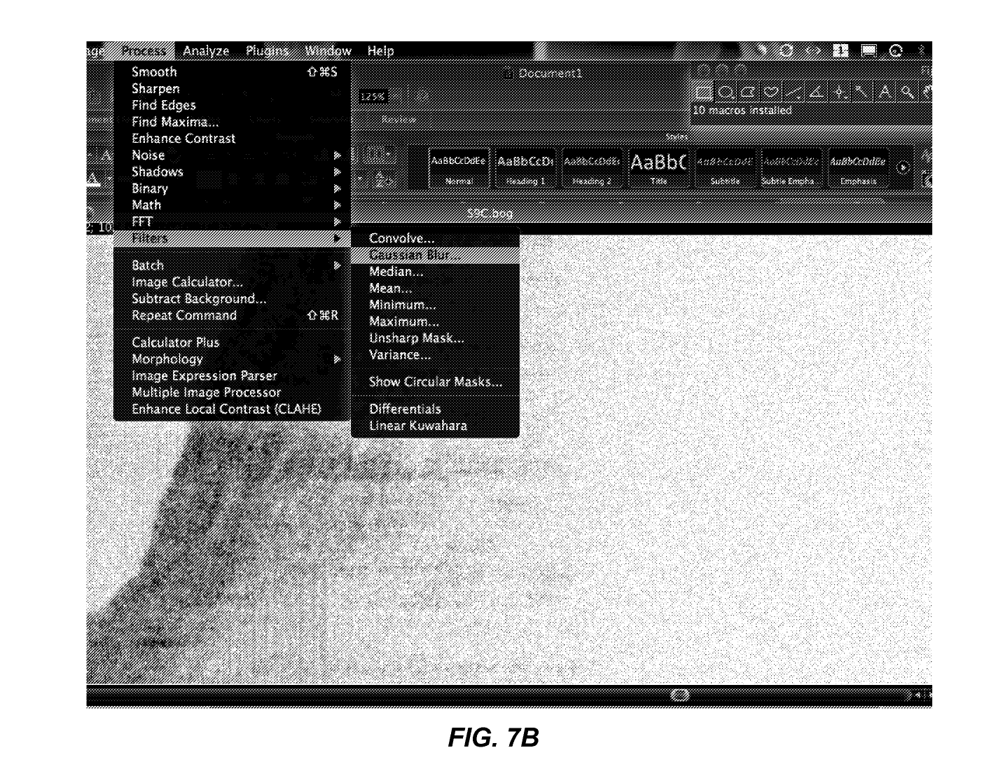

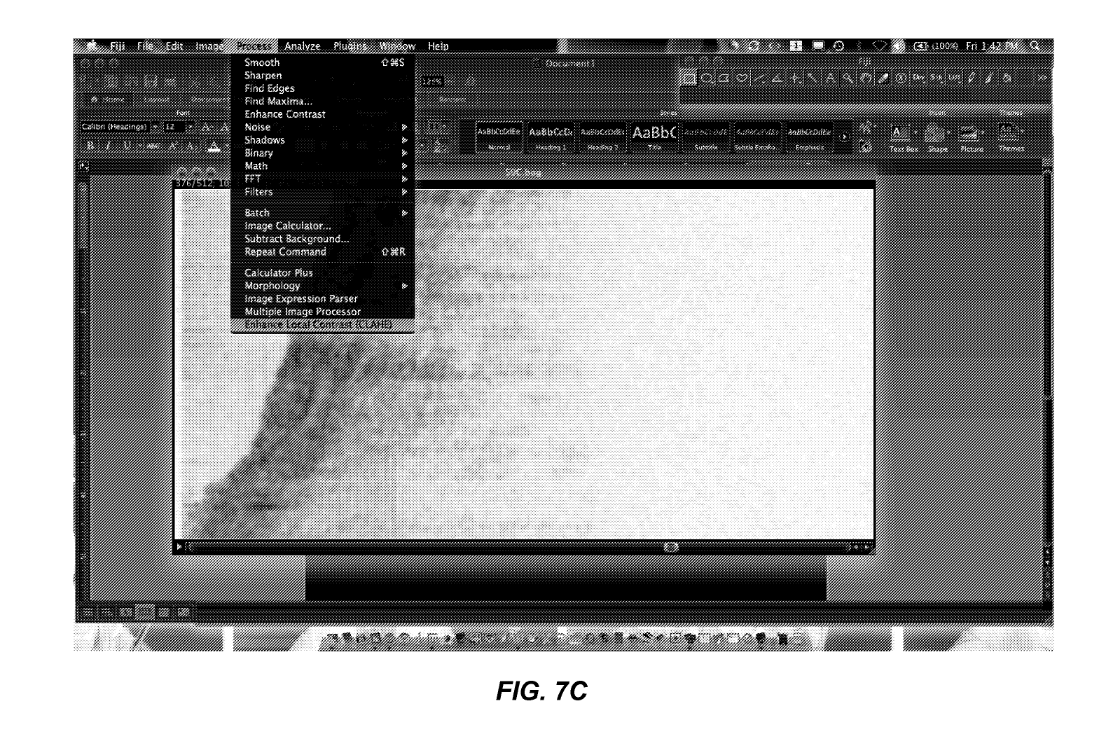

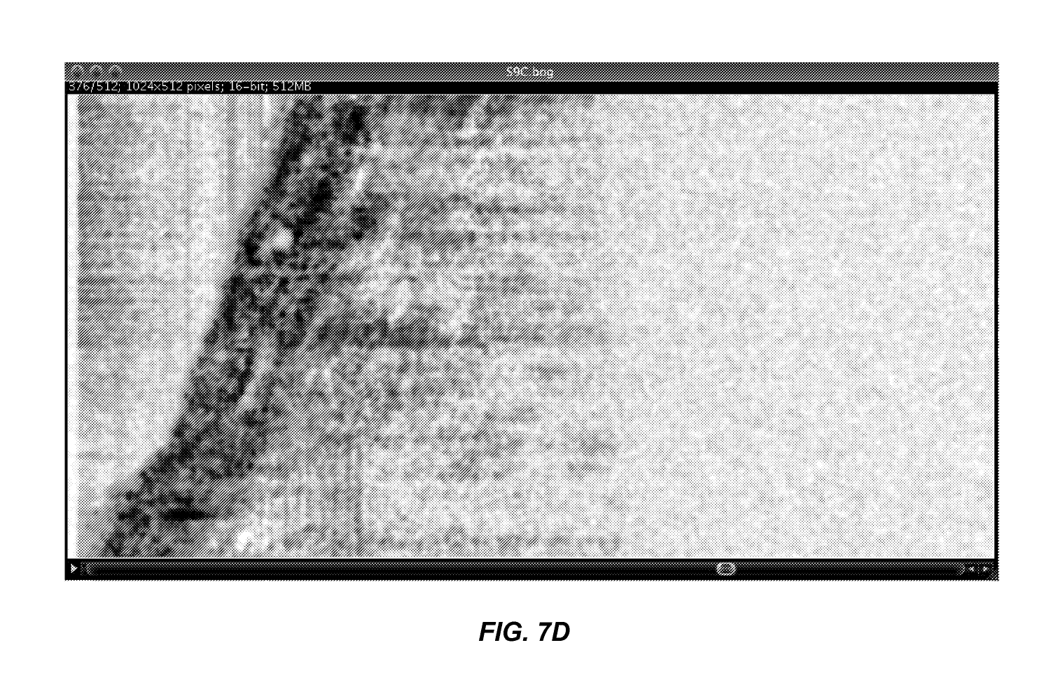

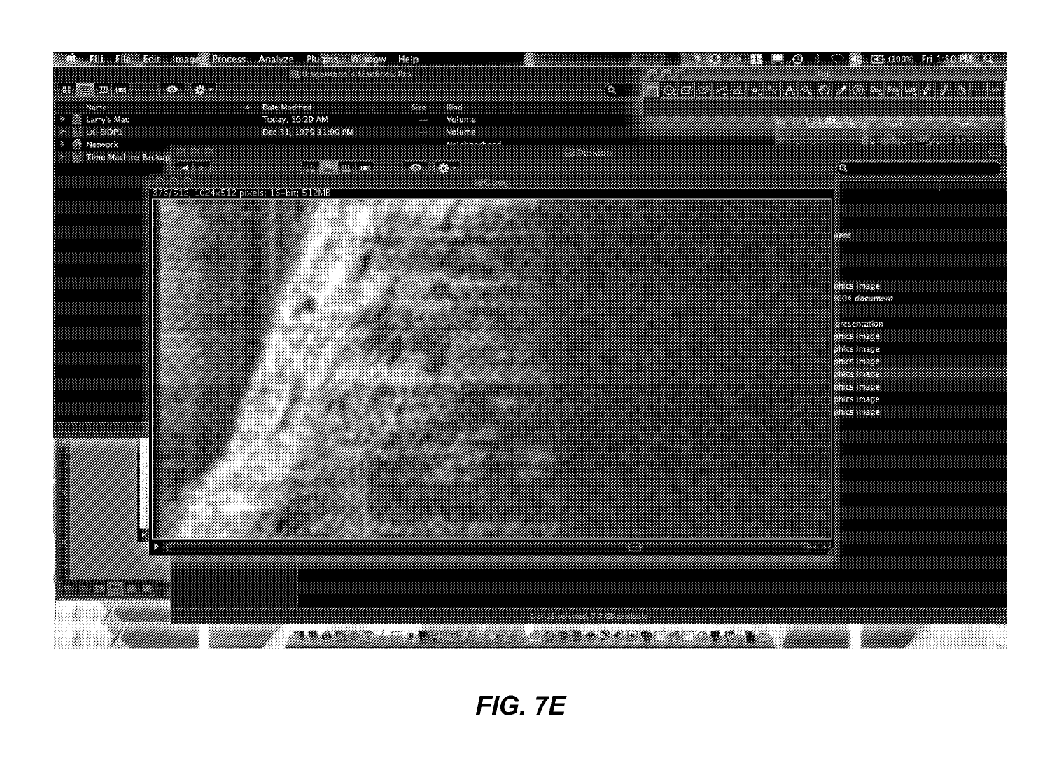

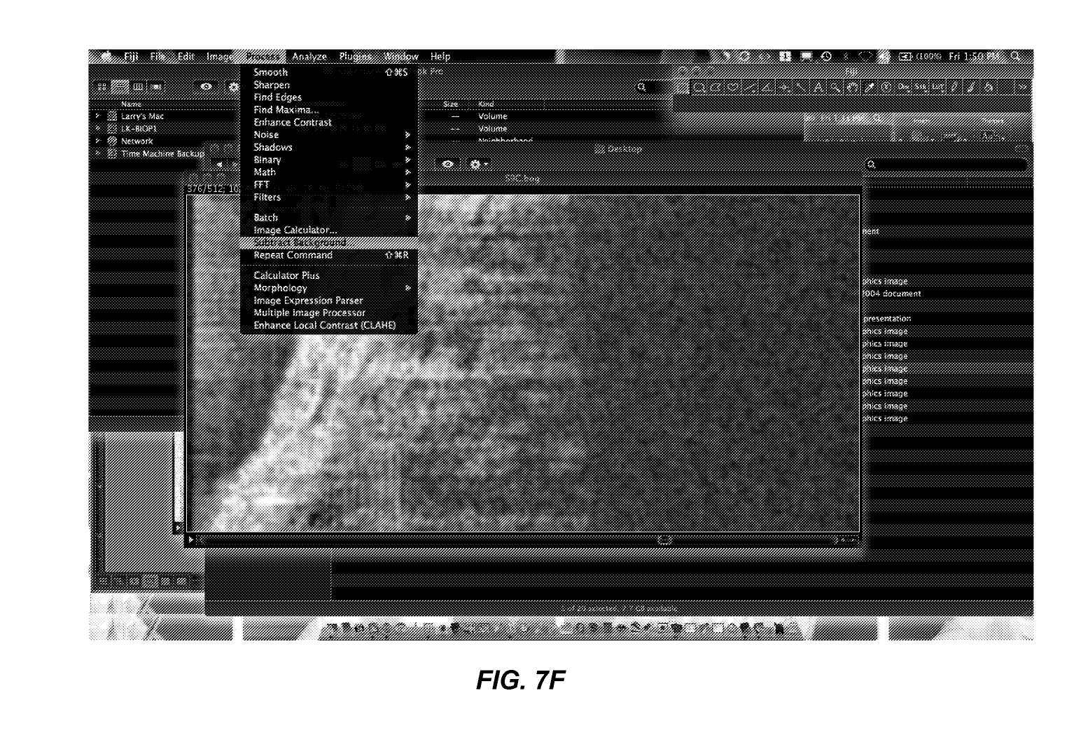

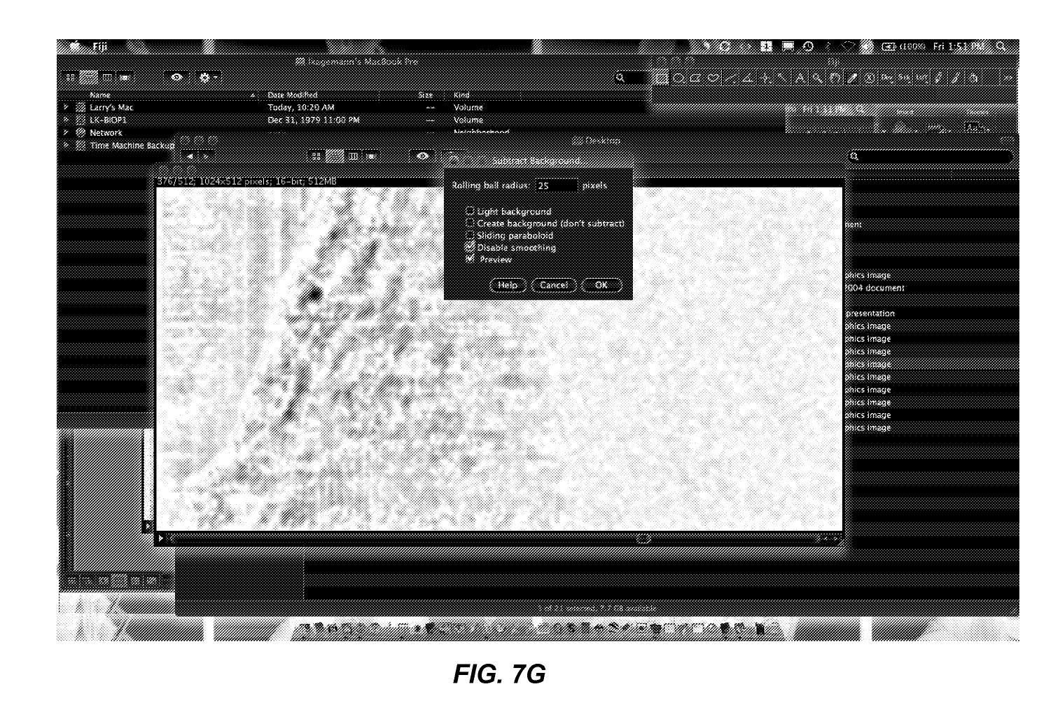

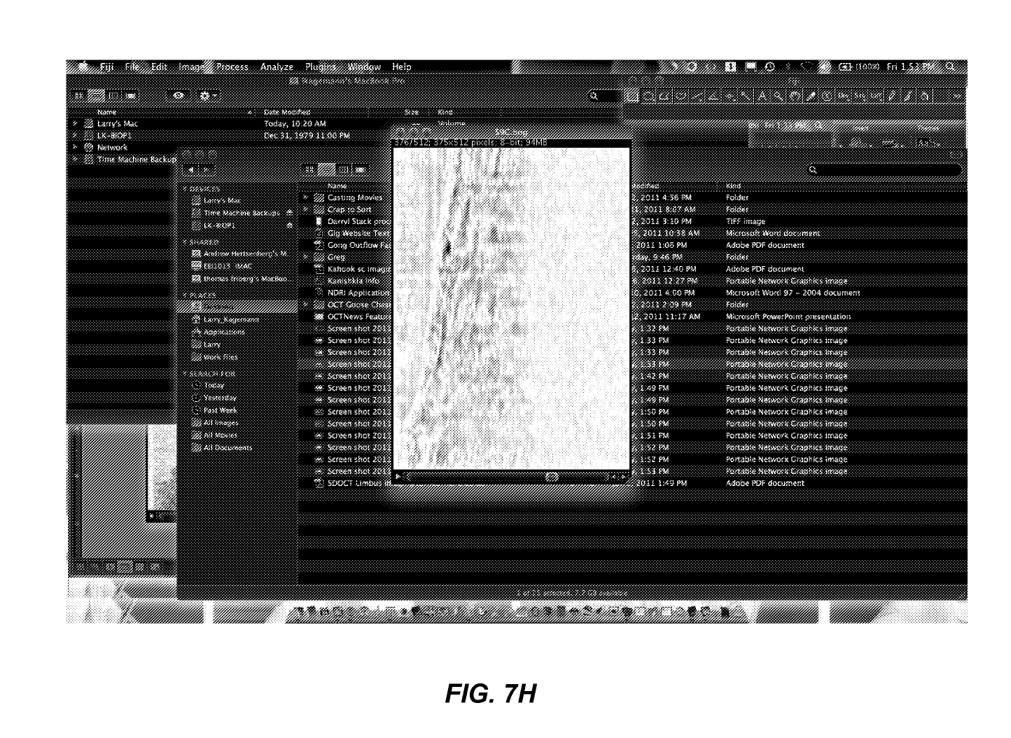

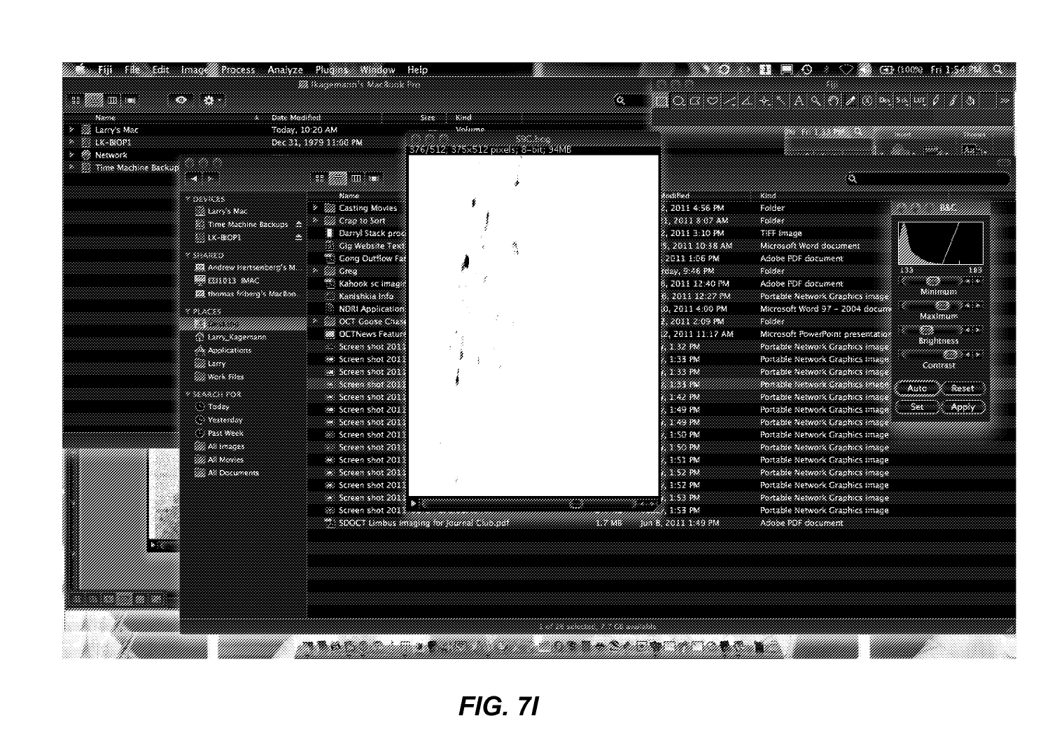

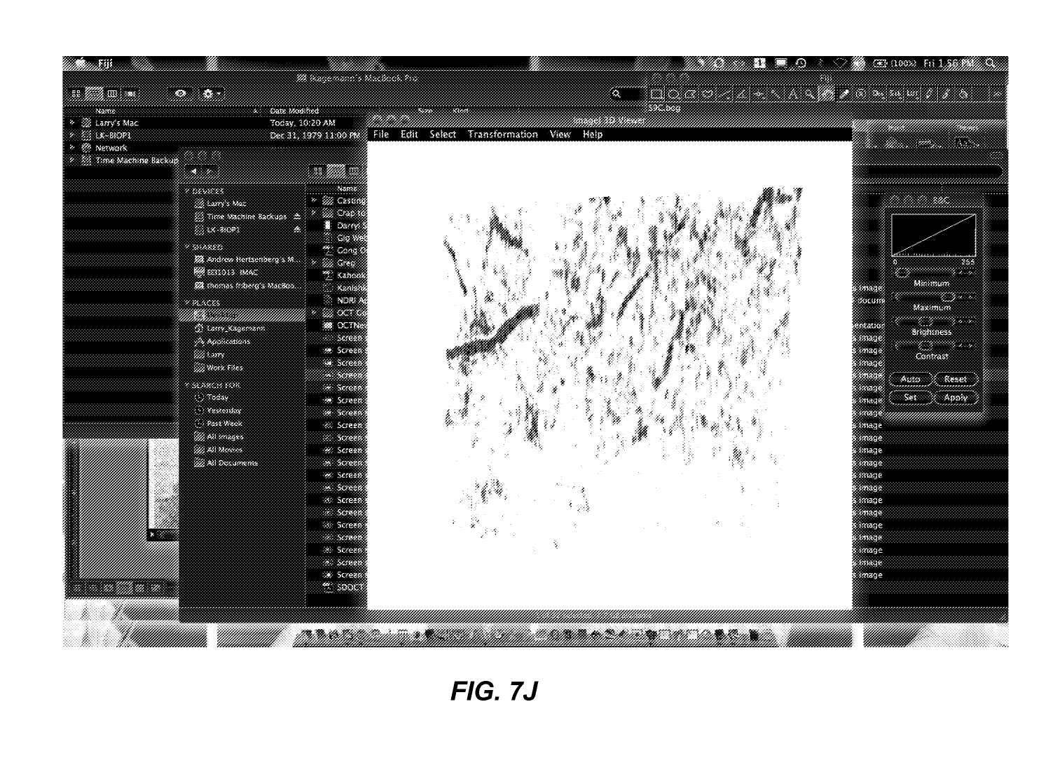

A working example of the image processing is illustrated in FIG. 7(A)-10(J).

The raw image was loaded into ImageJ FIJI (ImageJA ver 1.45; http://fiji.sc/wiki/index.php/Fiji) for processing. A single frame is shown in FIG. 7(A), though it is one of 512 sequential frames in a volumetric scan.

A Gaussian blur filter was run to remove some of the speckle noise, as illustrated in FIG. 7(B). FIG. 7(C) shows that the image intensity faded from left (near the scanning beam) to right (deep in the tissue). A contrast limited adaptive histogram equalization filter then was run to improve contrast of the visible open vessels and to increase visibility of deeper structures. This created an image with the appearance of FIG. 7(D).

The image was inverted black/white, with the result that the open vessels appeared as white structures in a dark background. See FIG. 7(E).

A Gaussian blur filter was run again, to remove speckles created by contrast enhancement, as illustrated in FIG. 7(F). The background was subtracted, using a rolling ball background subtraction routine with a radius of 25. The resulting image is shown in FIG. 7(G).

The image was converted to an 8-bit greyscale image and was resampled to obtain a 1:1:1 aspect ratio, as shown in Figure (H). Contrast was adjusted to blacken most of the image portions, except for the bright white vessels, as shown in Figure (I).

These volumetric data were displayed in 3D, yielding a vascular casting. See illustrated in FIG. 7(J).

Pursuant to the invention, functional SD-OCT imaging can be configured to obtain both structural and Doppler data simultaneously. For instance, each A-scan can be recorded multiple times before moving to the next location. The resulting data can be useful in locating open outflow structures that are not readily identified visually or subjectively. On the other hand, the Doppler data are not necessarily used to create the casting.

The total scan size was limited in the example system described presently; hence, multiple acquisitions of individual A-scans indicated that fewer A-scans can be obtained. In one instance, each location was scanned twice, including one round with a maximum number of A-scans (512×512), without Doppler data, used to create the casting; in another round, with 700×40 A-scans across the same location but with twelve Doppler repeats.

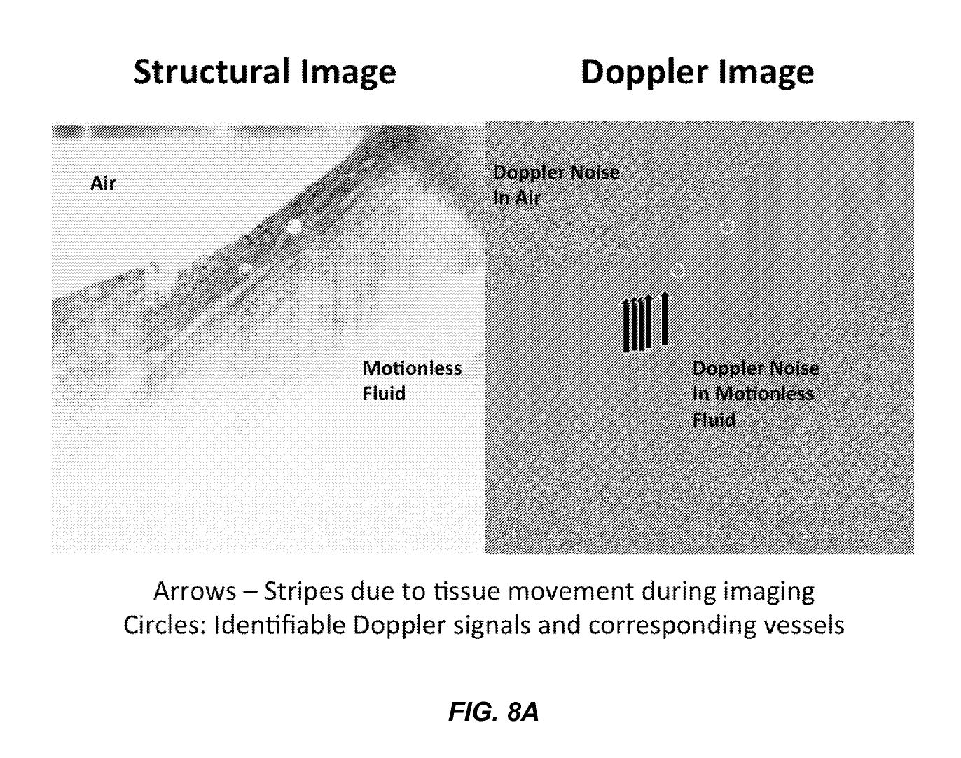

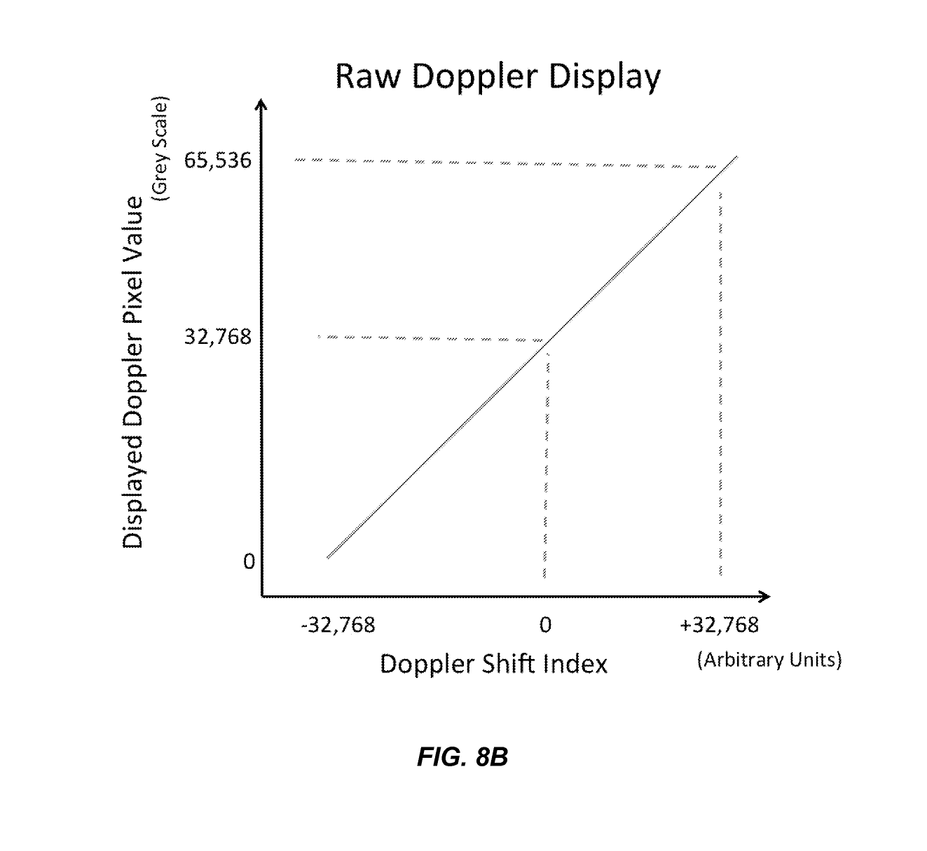

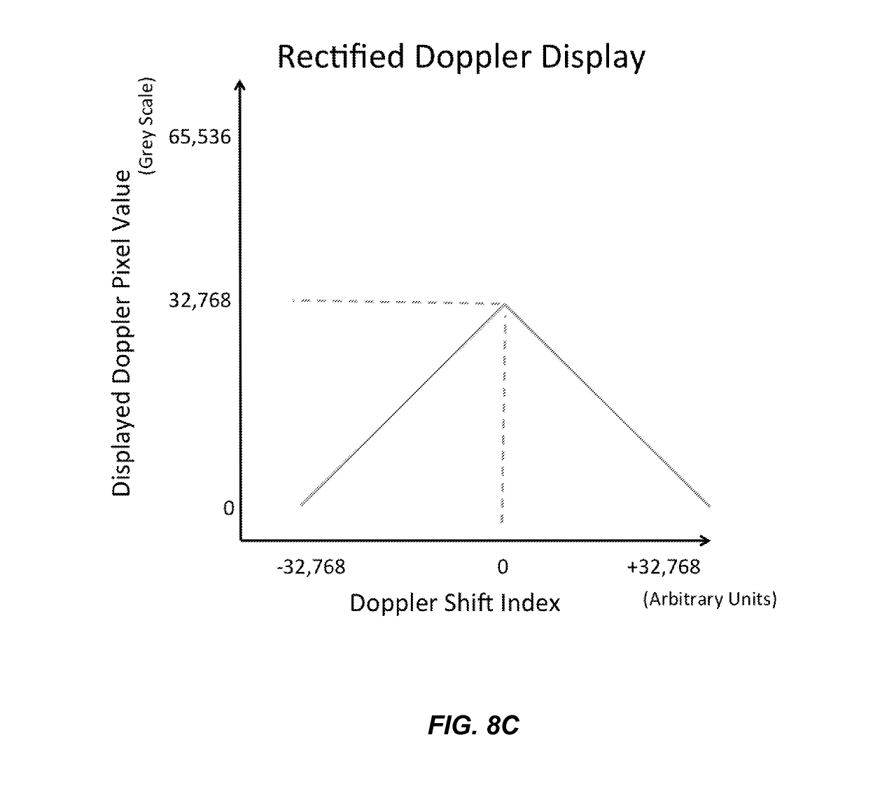

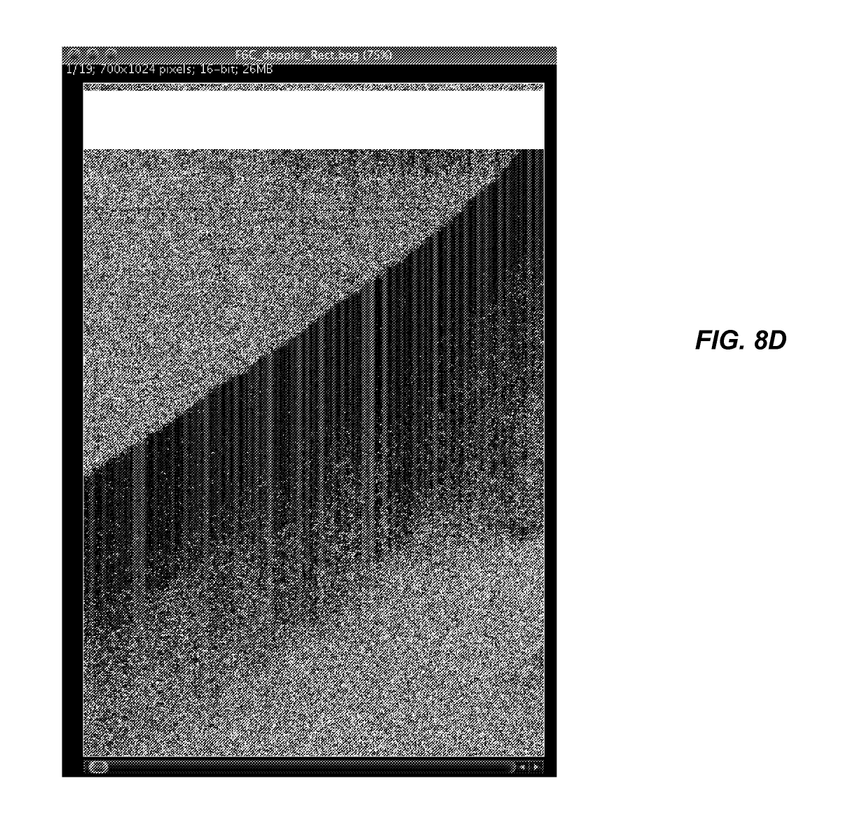

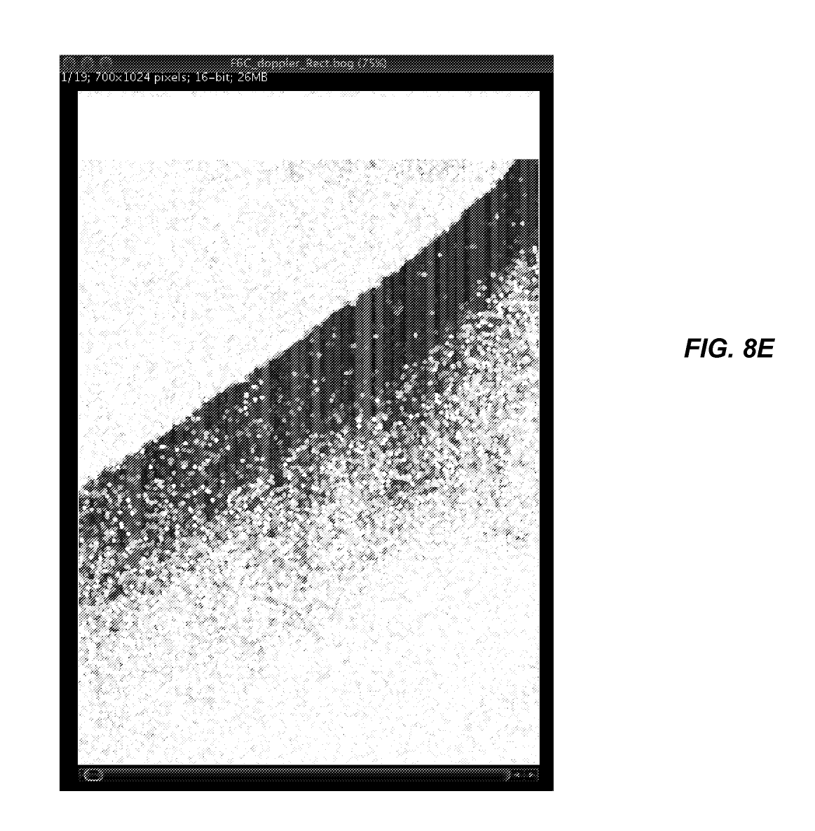

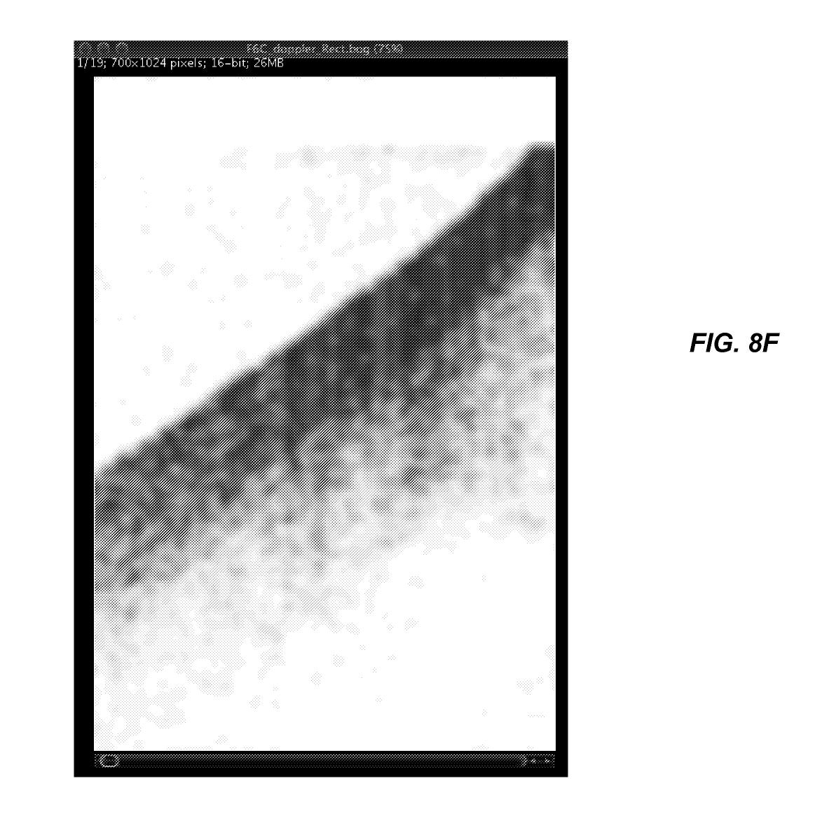

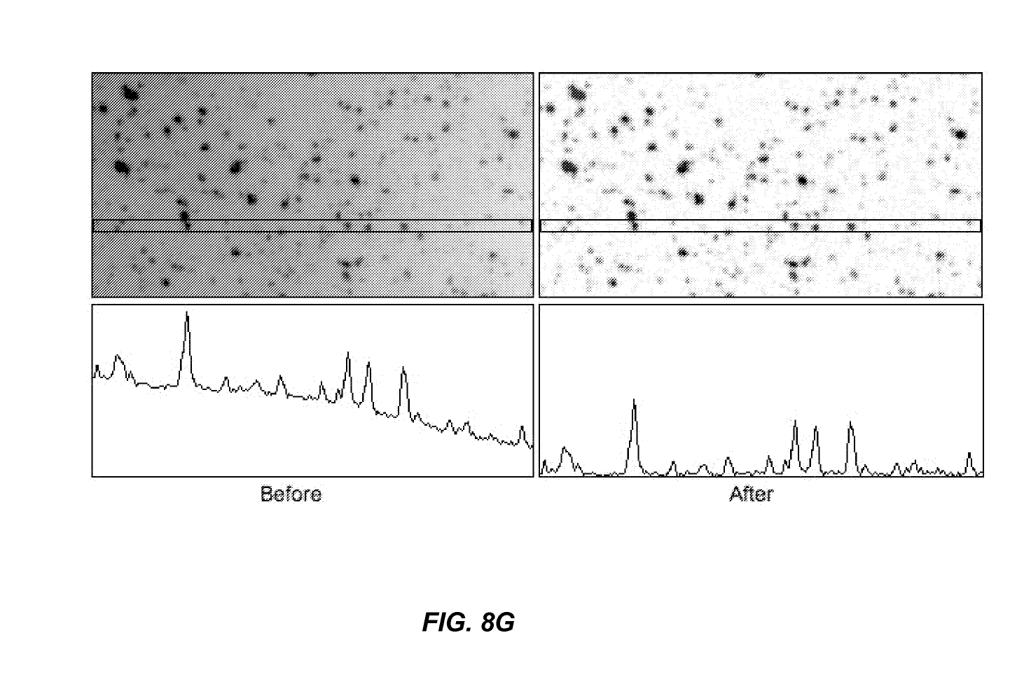

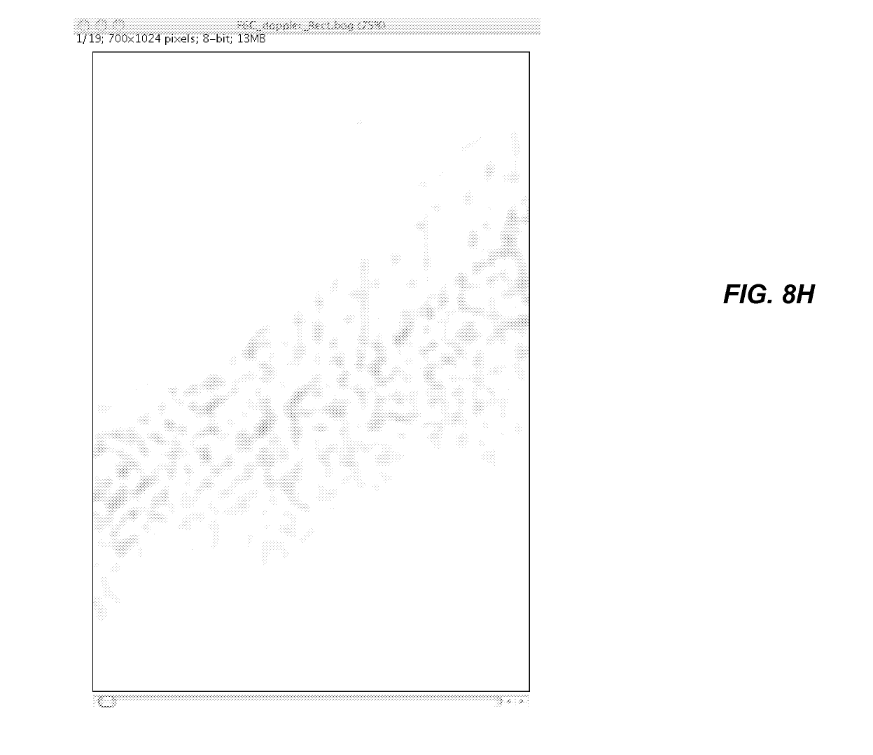

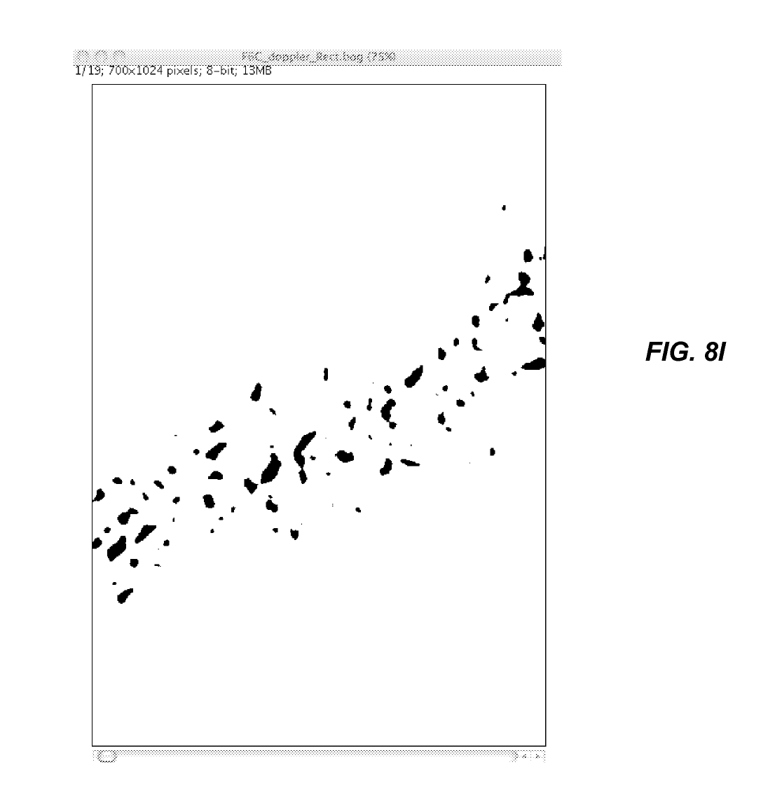

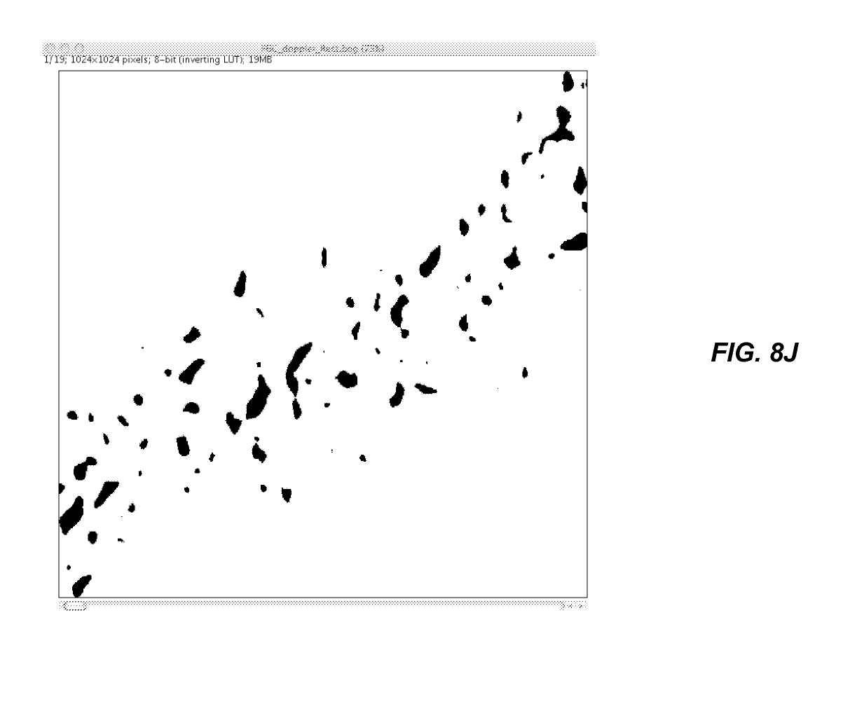

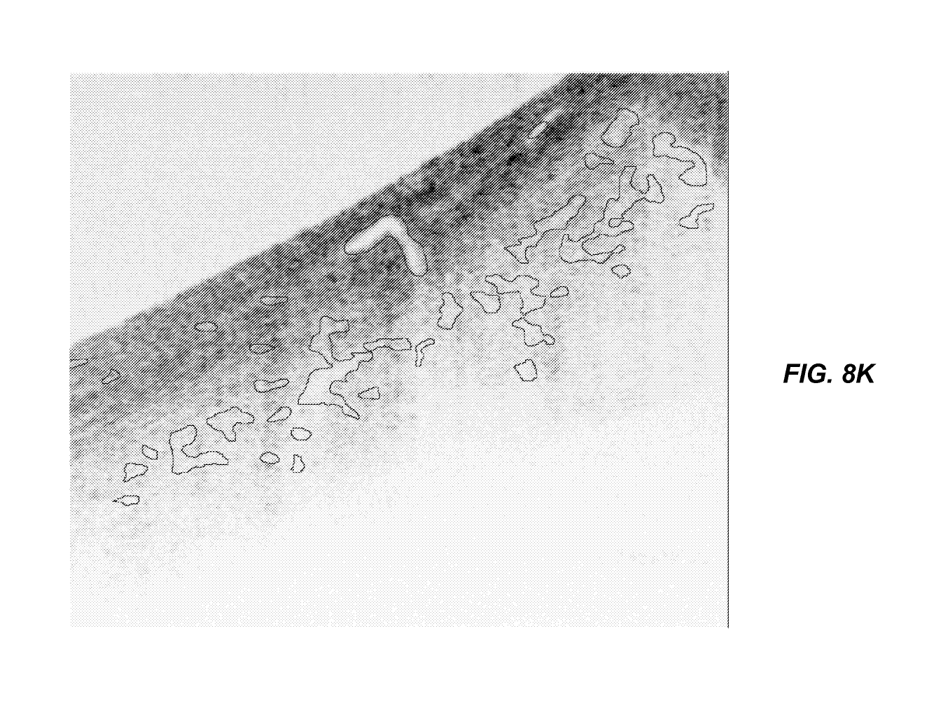

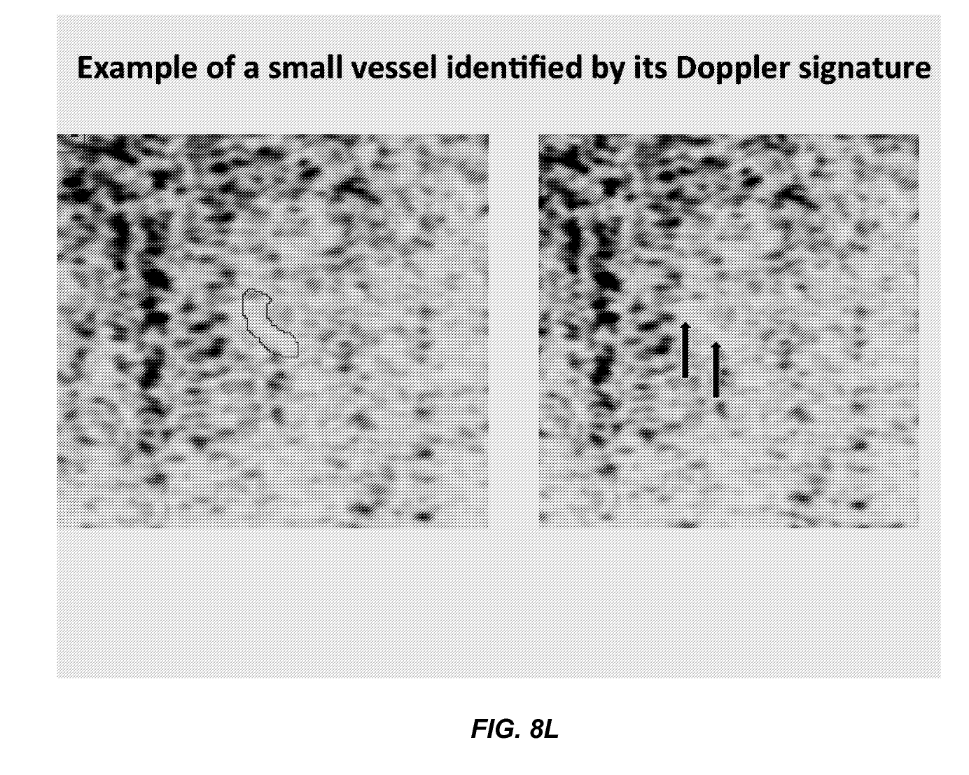

The foregoing technique can be employed to create paired images: one displaying tissue structure, and the other displaying Doppler shifts in the same space, as illustrated in FIG. 8(A). This image contains a segment of eye tissue exposed to the air, supported by fluid behind. In the Doppler image, both air and fluid are characterized by Gaussian noise. When stable tissue is present (light appearance in the structural image), its Doppler signal appears as solid grey. The pairing can be used to calculate a total flow, where the 512×512 casting is used for locating the areas that need to be measured, e.g., an area of interest, and the paired 700×40 images with twelve Doppler repeats are used to perform velocity measurements at the area of interest.

In this image, there was a motion artifact resulting in the tissue being imaged gently rocking back and forth toward and away from the beam, resulting in Doppler shifts toward the beam (positive Doppler shifts), appearing brighter than the background, and Doppler shifts away from the beam, displayed as dark (negative Doppler shifts, arrows). The small vessels (circles left) produce small Doppler signals that appear as light and dark pixels relative to the surrounding motionless tissue (circles right). Also, the strength of the Doppler signal within the image fades to noise as progressing through the tissue from the top to the bottom of the image.

FIG. 8(B) illustrates the relationship between the raw Doppler values within the image and their pixel values as displayed.

In accordance with the invention, locations of true motion sources within the Doppler image are automatically identified. Initially, as illustrated in FIG. 8(C), the Doppler image is rectified, in order that Doppler signals, regardless of their directions, appear darker than the surrounding tissue. Larger Doppler signals are darker, and smaller Doppler signals are less dark. This procedure results in a Doppler image as illustrated in FIG. 8(D).

The image then is eroded to minimum; specifically, pixels with a 3×3 pixel neighborhood are all set to the lowest value within that neighborhood. This results in an image with the appearance as illustrated in FIG. 8(E).

To remove residual artifact in the air and fluid regions, the image is gently blurred using a Gaussian blur with radius of 6 pixels. This produces an image with the appearance as illustrated in FIG. 8(F).

Background then is subtracted using Steinberg's “rolling ball” algorithm, which is discussed, for example, in “Biomedical Image Processing”, IEEE Computer, January 1983. The overall process, described at http://imagejdocu.tudor.lu/doku.php?id=gui:process:subtract_backgroundmm, is illustrated in FIG. 8(G). The resulting Doppler image is illustrated in FIG. 8(H).

The image then is made binary, using a histogram. Of the lightest pixels 97.5% are set to a value of white (255) and the remaining dark pixels set to a value of 0, as illustrated in. The resulting image is illustrated in FIG. 8(I).

The image then is resized to provide a 1:1 aspect ratio. Black regions with an area of 250 or more pixels in the corrected image are located, outlined, and their outlines recorded. The resulting image has an appearance as illustrated in FIG. 8(J).

FIG. 8(K) shows an illustrative image of vessels identified by Doppler signatures. FIG. 8(L) illustrates examples of small vessels identified by their Doppler signatures. The circle identifies the general location of the small vessels. The arrows point to the small vessels.

### WORKING EXAMPLE 2

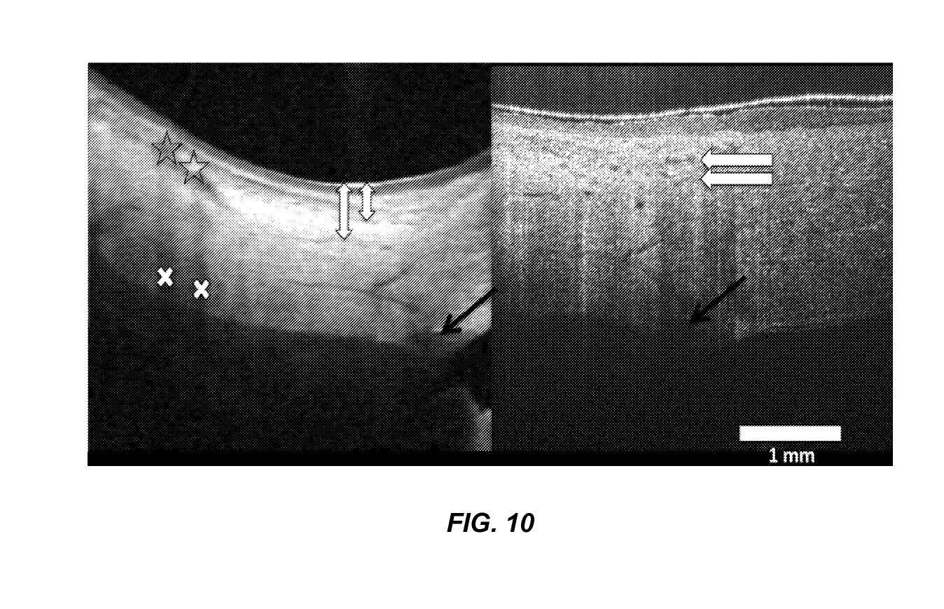

As described in further detail below, a commercially available, FDA-approved SD-OCT system can be used to produce a dataset amenable to processing, pursuant to the present invention, to achieve 3D visualization of the aqueous outflow system in living human eyes. Such visualization, according to the invention, can include features of the aqueous veins as well as Schlemm's canal (see FIG. 10, for example).

Implementation was as described above in Example 1, with these differences:
1. The data of the Cirrus system (see below) are 8-bit and do not require conversion (downsampling).
2. Before the steps described in Working Example 1, a 5×5×1 averaging kernel is applied: the “1” is the axis of the A-scan and the “5×5” represents five adjacent lateral positions in five adjacent frames or B-scans.
3. In this Example 2 each frame was cropped to include only the structure of interest. Frames (B-scans) surrounding the structure of interest were deleted. Only the locations and frames with the structure of interest were included in the visualization. This was done because noise and features surrounding the structure of interest cannot be removed automatically without degrading the quality of the structures of interest.

### Imaging of SC in Living Human Eyes

Six healthy volunteers were recruited from the staff and faculty of the University of Pittsburgh Medical Center Eye Center. Schlemm's canal and aqueous veins were imaged using two commercially available SD-OCT devices, the Cirrus HD-OCT (Carl Zeiss Meditec, Dublin, Calif.) and a modified Bioptigen SDOIS (Bioptigen, Research Triangle Park, N.C.). The Cirrus light source has a 50-nm bandwidth centered at 840 nm, resulting in a 5-μm coherence length in tissue. The Bioptigen optics engine was coupled with a quad diode light source with an 870-nm center wavelength and a 200-nm bandwidth (Q870, Superlum Ltd, Dublin, Ireland). The light source had a coherence length of 1.3 μm in tissue.

As in Working Example, 1, two scan protocols were used. The “volume” protocol optimized for the acquisition of 3D data (512×512 A-scans with no averaging; ˜9.4-second acquisition time), while the “2D slice” protocol optimized for visualization of individual frames (700×40 A-scans, each averaged 8 times at acquisition; ˜8-second acquisition time). Each protocol imaged a 4×4-mm transverse area of the limbus with a 2-mm A-scan length at the 3, 6, and 12 o'clock positions. Sequential scans in the volume protocol had a center-to-center spacing of 7.8 μm. With a lateral resolution of 20 μm, there was a high level of overlap (resampling) of tissue, with any single point within the scan volume being sampled by five axial scans (center plus nearest neighbors in the x-y plane). Sequential scans in the 2D slice protocol were separated by 100 μm. All scans were oriented so that the scan cube was tangential to the limbus and the center scan was radial to the limbus.

The commercially available Cirrus HD-OCT had two anterior segment scan protocols, both approved by the U.S. Food and Drug Administration (FDA). The 512×128 A-scan “cube” protocol (˜2.5-second scan time) was used, covering a 4×4-mm area of the limbus at 3, 6, and 12 o'clock. Unlike the Bioptigen, which was capable of orienting the B-scans at any arbitrary angle, only the 3 and 6 o'clock Cirrus B-scans had a radial orientation relative to the limbus; the 12 o'clock scan had a tangential orientation. Sequential frames in the Cirrus scans had a 31-μm center-to-center separation. Raw OCT signal data were exported from both devices.

### Image Processing

Scans were preprocessed and then were visualized in 3D using ImageJ Fiji (ImageJ 1.45k java, available at http://rsb.info.nih.gov/ij/). Cirrus scans were preprocessed with a 3×3×3 averaging kernel. More specifically, each voxel in the dataset was replaced by the average of 27 voxels in surrounding 3D space (3×3×3). Bioptigen volume images were preprocessed with a flat 5×5 averaging kernel. Each voxel was replaced with the average of the surrounding 5×5-voxel, 2D plane. Averaging protocols were selected subjectively based on the appearance of the outcome. Processing time for averaging was approximately one minute per image for both Bioptigen and Cirrus.

The Fiji “enhance local contrast” filter was used to improve visualization of structure throughout. To create virtual castings, images were inverted so that the black collector channels appeared as white structures. The “subtract background” filter was applied with a 30-pixel kernel. Images were resampled to provide a 1:1:1 voxel aspect ratio in 3D. Contrast was adjusted to isolate the collector channels and the volumes rendered using the 3D viewer plug-in. The total time to produce a 3D visualization was approximately 20 minutes, although multiple attempts to maximize visualization of structures were common. Varying levels of noise sources surrounding structures of interest necessitated flexibility in the degree to which noise was suppressed, to minimize noise content with minimal loss of image content.

Two distinct layers of aqueous venous plexuses were subjectively identified in the 2D visualizations (FIG. 10). The distance from the surface of the limbus to each aqueous venous plexus was measured in the 2D slices in nasal, inferior, temporal, and superior quadrants. The diameters of aqueous veins in each layer of the aqueous venous plexus were measured in each quadrant. No fewer than six of the forty frames of the 2D scan protocol were included for each location when venous plexus depth and aqueous vein diameter were assessed.

### Results

Four men and two women (average age, 38.5 and 29 years, respectively) were imaged on two different days. As shown in FIG. 10, outflow pathways were readily visible in both Cirrus and Bioptigen images from all eyes. The 2D slices revealed both outflow and vascular structures. Blood vessels cast vertical shadows in OCT images. Shadows were useable to identify blood vessels. Aqueous veins were identified by tracing their pathway to SC, and their identity as aqueous veins was confirmed by the lack of shadows. Table 1 presents the average depth of the superficial and deep aqueous venous plexuses and the average aqueous vein diameters.

Acquisition of 3D volumetric samples enabled identification of the same location within the limbus in both scan sets, based on subjective observation of outflow pathway morphology (see FIG. 10). Speckle noise obscured visualization of SC and aqueous veins within the raw Cirrus scans, but 3×3×3 averaging reduced speckle and allowed clear visualization. Utilizing adjacent frames, the pathway from SC to the midlimbal intrascleral plexus could be traced.

TABLE 1
Aqueous Venous Plexus Characteristics

| | Mean (SD) Distance from | |
|---|---|---|
| | Surface to Aqueous | Mean (SD) Aqueous |
| Location | Venous Plexus (μm) | Vein Diameter (μm) |
| Nasal superficial | 133.17 (23.29) | 38.28 (7.22) |
| Inferior superficial | 95.94 (12.44) | 33.03 (8.72) |
| Temporal superficial | 115.30 (14.08) | 32.88 (9.23) |
| Superior superficial | 99.50 (52.71) | 18.07 (8.58) |
| Nasal deep | 295.61 (59.72) | 48.53 (9.05) |
| Inferior deep | 318.35 (68.81) | 74.42 (20.56) |
| Temporal deep | 258.58 (51.77) | 39.46 (6.13) |
| Superior deep | 270.55 (132.67) | 34.22 (16.49) |

SD = standard deviation.
The distance between the surface of the limbus and each of the 2 layers of aqueous venous plexuses was measured in each quadrant. The diameter of aqueous veins within those plexuses also was measured.

With the data produced on each system, virtual casting was feasible of the aqueous humor outflow structures between SC and the episcleral vasculature, as well as of surrounding blood vessels, the identity of which was suggested by their relatively large size. The degree to which noise was suppressed altered the image content. Leaving more noise allowed visualization of aqueous outflow microvasculature with a “fishnet” appearance. Removal of more noise eliminated visualization of the aqueous humor microvasculature, leaving only large blood vessels in the casting.

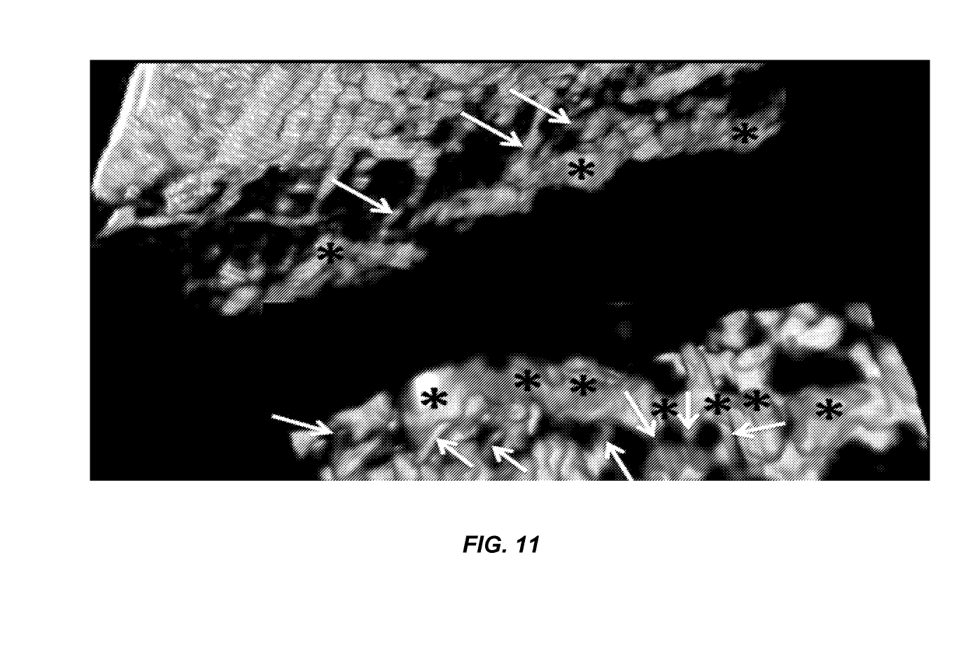

FIG. 11 presents virtual castings of SC (asterisks) in the living human eye. Several collector channels (arrows) and aqueous veins extending from SC were observed within the 4-mm section of SC. When the 2D image stack that produced these castings was reviewed, the presence of four aqueous vein branch points was not evident. The SC could be cast from all Cirrus scans at both nasal and temporal quadrants. When the castings were rotated in 3D space, collector channels emanating from SC could be observed in nine of the twelve castings.

Pursuant to the invention, therefore, a virtual casting of AH outflow structures can be achieved non-invasively. The imaging thus obtained, pursuant to the invention, demonstrates that clinically useful, direct assessment of outflow in patients with glaucoma can be obtained, likewise based on the virtual casting. For instance, employing the invention should be feasible to evaluate, prognostically as well as diagnostically, the structural integrity and general status of the Schlemm's canal, morphologic changes in which are believed associated with acute elevation of IOP, a primary risk factor for primary open-angle glaucoma. See Kagemann et al., 2012.

A method of the invention for estimating in vivo aqueous humor outflow for a subject thus can include (1) preparing a virtual casting of the outflow network of said subject by use of background subtraction and contrast enhancement; (2) tracing network terminal branches in said reduced virtual casting; (3) obtaining Doppler data for at least some of said terminal branches, to calculate a fluid velocity within each of such terminal branch; then (4) pairing each fluid velocity for each such terminal branch with a measurement of cross-sectional area for that terminal branch, thereby to provide a plurality of volumetric flow values; and (5) integrating over said plurality to obtain a volumetric estimate of aqueous humor outflow for the subject.

The preparing of a virtual casting can include oversampling a tissue volume in the eye with multiple scans. It also can include reducing speckle noise and improving contrast of the network terminal branches against background tissue by averaging the multiple scans or by using a small-neighborhood Gaussian blur filter. Further, the virtual casting process can include spectral domain optical coherence tomography scanning, as discussed above.

In accordance with one embodiment of the invention, preparing a virtual casting includes imposing a connectivity requirement for the network terminal branches for inclusion in the virtual casting. Additionally, a virtual casting process can entail selectively constructing a subset of the pathway based on the fluid velocity. The tracing can involve visual identification or, alternatively, automatic identification by means of an artificial intelligence method. Suitable applications of artificial intelligence for pattern recognition are known, as exemplified by the method for pattern recognition and feature classification described in U.S. Pat. No. 5,325,445, the entire contents of which is incorporated here by reference.

Pursuant to the invention, preparation of a virtual casting can comprise forming paired images: a first image, displaying tissue structure at a location of the subject; and a second image displaying Doppler shifts at the same location.

The invention also contemplates apparatus that includes a processor, to process scanned images of a subject, that is configured to (A) process data including both structural and flow velocity information of the subject, (B) construct from the structural information a virtual casting of a primary aqueous humor outflow pathway of the subject, and (C) identify terminal branches of said primary aqueous humor outflow pathway from said virtual casting. In a preferred embodiment, the flow velocity information is obtained from Doppler measurements, thereby to obtain the aforementioned flow velocity information. The processor can be configured as well to calculate a flow rate in the aqueous humor outflow pathway. The processor also can be configured to determine a total aqueous humor outflow by integrating the flow rate in the identified terminal branches.

In accordance with another aspect of the invention, a system is provided that includes a spectral domain optical coherence tomography scanner and a processor to process data obtained from the scanner. The processor is configured (A) to process data that include both structural and flow velocity information of the subject, (B) to construct, from the structural information, a virtual casting of a primary aqueous humor outflow pathway of the subject, and (C) to identify terminal branches of the primary aqueous humor outflow pathway from the virtual casting. In this regard, the invention encompasses a non-transitory computer readable medium having instructions stored thereon for: (1) preparing a virtual casting of the outflow network of the subject by use of background subtraction and contrast enhancement; (2) tracing network terminal branches in the resultant, noise-reduced virtual casting; (3) obtaining Doppler data for at least some of the terminal branches, thereby to calculate a fluid velocity within each of such terminal branches; then (4) pairing each fluid velocity for each terminal branch with a measurement of cross-sectional area for that terminal branch, providing a plurality of volumetric flow values; and (5) integrating over that plurality to obtain a volumetric estimate of aqueous humor outflow for the subject.

### Cited Publications

Ascher, K. W., 1942. Aqueous veins: physiologic importance of visible elimination of intraocular fluid. Am J Ophthalmol 25.

Ashton, N., 1951. Anatomical study of Schlemm's canal and aqueous veins by means of neoprene casts. Part I. Aqueous veins. Br J Ophthalmol 35, 291-303.

Ashton, N., 1952. Anatomical study of Schlemm's canal and aqueous veins by means of neoprene casts. II. Aqueous veins. Br J Ophthalmol 36, 265-267; contd.

Ashton, N., Smith, R., 1953. Anatomical study of Schlemm's canal and aqueous veins by means of neoprene casts. III. Arterial relations of Schlemm's canal. Br J Ophthalmol 37, 577-586.

Barany, E. H., 1964. Simultaneous Measurement of Changing Intraocular Pressure and Outflow Facility in the Vervet Monkey by Constant Pressure Infusion. Invest Ophthalmol 3, 135-143.

Battista, S. A., Lu, Z., Hofmann, S., Freddo, T., Overby, D. R., Gong, H., 2008. Reduction of the available area for aqueous humor outflow and increase in meshwork herniations into collector channels following acute IOP elevation in bovine eyes. Invest Ophthalmol Vis Sci 49, 5346-52.

Cense, B., Chen, T. C., Nassif, N., Pierce, M. C., Yun, S. H., Park, B. H., Bouma, B. E., Tearney, G. J., de Boer, J.F., 2006. Ultra-high speed and ultra-high resolution spectral-domain optical coherence tomography and optical Doppler tomography in ophthalmology. Bull Soc Belge Ophtalmol, 123-32.

Dielemans, I., Vingerling, J. R., Wolfs, R. C., Hofman, A., Grobbee, D. E., de Jong, P. T., 1994. The prevalence of primary open-angle glaucoma in a population-based study in The Netherlands. The Rotterdam Study. Ophthalmology 101, 1851-1855.

Duke-Elder, S., 1949. The physiology of the intraocular fluids and its clinical significance. Trans Am Acad Ophthalmol Otolaryngol 54, 18-25.

Ellingsen, B. A., Grant, W. M., 1971. The relationship of pressure and aqueous outflow in enucleated human eyes. Invest Ophthalmol 10, 430-437.

Erickson-Lamy, K., Rohen, J. W., Grant, W. M., 1991. Outflow facility studies in the perfused human ocular anterior segment. Exp Eye Res 52, 723-31.

Hann, C. R., Bentley, M. D., Vercnocke, A., Ritman, E. L., Fautsch, M. P., 2011. Imaging the human aqueous humor outflow pathway in human eyes by three-dimensional micro-computed tomography (3D micro-CT). Exp Eye Res. 92, 104-111.

Irshad, F. A., Mayfield, M. S., Zurakowski, D., Ayyala, R. S., 2010. Variation in Schlemm's canal diameter and location by ultrasound biomicroscopy. Ophthalmology 117, 916-920.

Johnstone, M. A., 2004. The aqueous outflow system as a mechanical pump: evidence from examination of tissue and aqueous movement in human and non-human primates. J Glaucoma 13, 421-438.

Kagemann, L., Wollstein, G., Ishikawa, H., Bilonick, R. A., Brennen, P. M., Folio, L. S., Gabriele, M. L., Schuman, J. S., 2010. Identification and assessment of Schlemm's canal by spectral-domain optical coherence tomography. Invest Ophthalmol Vis Sci 51, 4054-4059.

Kagemann, L., Wollstein, G., Ishikawa, H., Bilonick, R. A., Sigal, I. A., Nevins, J. E., Nadler, Z., Grimm, J., Jan, N.-J., Shuman, J. S., 2012. Schlemm's Canal (SC) narrowing in response to intraocular pressure (IOP) elevation observed by Spectral Domain Optical Coherence Tomography. IOVS 2012, Volume 53, ISIE E-Abstract, Program #P19.

Kahn, H. A., Leibowitz, H. M., Ganley, J. P., Kini, M. M., Colton, T., Nickerson, R. S., Dawber, T. R., 1977. The Framingham Eye Study. I. Outline and major prevalence findings. Am J Epidemiol 106, 17-32.

Kass, M. A., Hart, W. M., Jr., Gordon, M., Miller, J. P., 1980. Risk factors favoring the development of glaucomatous visual field loss in ocular hypertension. Surv. Ophthalmol. 25, 155-162.

Kymes, S. M., Plotzke, M. R., Li, J. Z., Nichol, M. B., Wu, J., Fain, J., The increased cost of medical services for people diagnosed with primary open-angle glaucoma: a decision analytic approach. Am. J. Ophthalmol. 150, 74-81.

Millar, C., Kaufman, P., 1995. Aqueous humor: secretion and dynamics in Tasman, W., Jaeger E A. Eds, Duane's foundations of clinical ophthalmology. Lippincott-Raven, Pa.

Quigley, H. A., Broman, A. T., 2006. The number of people with glaucoma worldwide in 2010 and 2020. Br J Ophthalmol 90, 262-267.

Reynolds, D. C., 1977. Relative risk factors in chronic open-angle glaucoma: an epidemiological study. Am J Optom Physiol Opt 54, 116-120.

Rollins, A., Yazdanfar, S., Kulkarni, M., Ung-Arunyawee, R., Izatt, J., 1998. In vivo video rate optical coherence tomography. Opt Express 3, 219-229.

Sarunic, M. V., Asrani, S., Izatt, J. A., 2008. Imaging the ocular anterior segment with realtime, full-range Fourier-domain optical coherence tomography. Arch Ophthalmol 126, 537-542.

Sommer, A., 1989. Intraocular pressure and glaucoma. Am J Ophthalmol 107, 186-188.

Stein, D. M., Ishikawa, H., Hariprasad, R., Wollstein, G., Noecker, R. J., Fujimoto, J. G., Schuman, J. S., 2006. A new quality assessment parameter for optical coherence tomography. Br J Ophthalmol 90, 186-190.

Vacharat, N., 1979. Risk factors in glaucoma. Int Ophthalmol Clin 19, 157-197. van der Merwe, E. L., Kidson, S. H., 2010. Advances in imaging the blood and aqueous vessels of the ocular limbus. Exp Eye Res 91, 118-126.

Zhang, K., Kang, J. U., 2010. Real-time 4D signal processing and visualization using graphics processing unit on a regular nonlinear-k Fourier-domain OCT system. Opt Express 18, 11772-84.

What is claimed is:

1. A method for estimating in vivo aqueous humor outflow for a subject, comprising the steps of:
(A) preparing a virtual casting of the outflow network of said subject by use of background subtraction and contrast enhancement;
(B) tracing network terminal branches in said virtual casting;
(C) obtaining Doppler data for at least some of said terminal branches, to calculate a fluid velocity within each of such terminal branches; then
(D) pairing each fluid velocity for each of such terminal branches with a measurement of cross-sectional area for that terminal branch, thereby to provide a plurality of volumetric flow values; and
(E) integrating over said plurality to obtain a volumetric estimate of aqueous humor outflow for said subject.

2. The method of claim 1, wherein said preparing a virtual casting comprises oversampling a tissue volume in the eye with multiple scans.

3. The method of claim 1, wherein said preparing a virtual casting comprises a spectral domain optical coherence tomography scanning

4. The method of claim 1, wherein said preparing a virtual casting comprises reducing speckle noise and improving a contrast of the network terminal branches against background tissue by averaging the multiple scans or by using a small-neighborhood Gaussian blur filter.

5. The method of claim 1, wherein said preparing of a virtual casting comprises imposing a connectivity requirement for the network terminal branches for inclusion in said virtual casting.

6. The method of claim 1, wherein said preparing of a virtual casting comprises selectively constructing a subset of said at least some of said terminal branches based on said fluid velocity.

7. The method of claim 1, wherein said tracing comprises visually identifying said network terminal branches.

8. The method of claim 1, wherein said tracing comprises identifying said network terminal branches via an automated artificial intelligence method.

9. The method of claim 1, further comprising forming paired images including (i) a first image that displays tissue structure at a location of the subject and (ii) a second image that displays Doppler shifts at the same location.

10. The method of claim 1, further comprising locating an area of interest from said virtual casting, wherein said integrating is within said area of interest.

11. An apparatus comprising a processor to process scanned images of a subject, wherein the processor is configured to:
(A) process data including both structural and flow velocity information of the subject;
(B) construct, from said structural information, a virtual casting of a primary aqueous humor outflow pathway of the subject; and
(C) identify terminal branches of said primary aqueous humor outflow pathway from said virtual casting.

12. The apparatus of claim 11, wherein said flow velocity information is obtained from Doppler measurements to obtain said flow velocity information.

13. The apparatus of claim 12, wherein the processor is further configured to calculate a flow rate in the aqueous humor outflow pathway.

14. The apparatus of claim 13, wherein the processor is further configured to determine a total aqueous humor outflow by integrating said flow rate in said identified terminal branches.

15. A system comprising a spectral domain optical coherence tomography scanner and a processor to process data obtained from said spectral domain optical coherence tomography scanner, wherein the processor is configured to:
(A) process data including both structural and flow velocity information of the subject;
(B) construct from said structural information a virtual casting of a primary aqueous humor outflow pathway of the subject; and
(C) identify terminal branches of said primary aqueous humor outflow pathway from said virtual casting.

16. A non-transitory, computer-readable medium having instructions stored thereon, wherein the instructions comprise:
(A) preparing a virtual casting of the outflow network of said subject by use of background subtraction and contrast enhancement;
(B) tracing network terminal branches in said reduced virtual casting;
(C) obtaining Doppler data for at least some of said terminal branches, to calculate a fluid velocity within each of such terminal branch; then
(D) pairing each fluid velocity for each such terminal branch with a measurement of cross-sectional area for that terminal branch, thereby to provide a plurality of volumetric flow values; and
(D) integrating over said plurality to obtain a volumetric estimate of aqueous humor outflow for said subject.
